(19) United States
(12) Patent Application Publication
(10) Pub. No.: US 2009/0017164 A1
(43) Pub. Date: Jan. 15, 2009

(54) FERMENTATION PROCESS FOR THE PREPARATION OF ETHANOL FROM A CORN FRACTION HAVING LOW OIL CONTENT

(75) Inventors: David Owen Schisler, Oskaloosa, IA (US); MaryJo Zidwick, Wayzata, MN (US); Mark Powers, Plymouth, MN (US); Mary M. Lazio, Ottumwa, IA (US); Jeffrey D. Ulku, Orono, MN (US); Neal Jakel, Janesville, WI (US)

Correspondence Address: SENNIGER POWERS LLP, 100 NORTH BROADWAY, 17TH FLOOR, ST LOUIS, MO, 63102, US

(73) Assignees: RENESSEN LLC, Creve Coeur, MO (US); CARGILL, INC., Wayzata, MN (US)

(21) Appl. No.: 12/029,054

(22) Filed: Feb. 11, 2008

Related U.S. Application Data
(60) Provisional application No. 60/945,195, filed on Jun. 20, 2007.
(60) Provisional application No. 60/889,602, filed on Feb. 13, 2007.

(51) Int. Cl. C12C 11/00; C12P 7/06

(52) U.S. Cl. 426/62; 435/165

(57) ABSTRACT

An improved fermentation process for the preparation of ethanol and distillers dried grain from a corn fraction having low oil, high starch and low germ content is provided. The process provides reduced fermentation time, increased ethanol yield and reduced fusel oil concentration.

This application claims the benefit of U.S. provisional application Ser. Nos. 60/889,602 (filed Feb. 13, 2007) and 60/945,195 (filed Jun. 20, 2007), the entire contents of which are incorporated herein by reference.

### FIELD OF THE INVENTION

The present invention generally relates to improved processes for the preparation of ethanol and distillers dried grain by fermentation of a corn fraction having low oil, high starch and low germ content.

### BACKGROUND OF THE INVENTION

Corn, Zea mays, is grown for many reasons including its use in food and industrial applications. Ethanol, distillers dried grain (DDG) and distillers dried grain with solubles (DDGS) are some of many useful products derived from corn.

The demand for ethanol and biodiesel is expected to increase as sources of oil based transportation fuels are depleted and replacements for those fuels are sought in an effort to reduce the impact of carbon dioxide based global warming. A significant portion of that demand will be met by increasing the amount of ethanol prepared from corn. Due to the commercial interest in fuel ethanol, there is a continued need for improvement in ethanol manufacturing processes which include a fermentation step.

### SUMMARY OF THE INVENTION

Among the various aspects of the present invention is the provision of improved fermentation processes for preparing ethanol from corn, the provision of such an improved processes in which the low oil fraction of fractionated corn is used as a fermentation feedstock, and the provision of dried distillers grain with solubles (DDGS) compositions having high protein content.

Briefly, therefore, one aspect of the present invention is a fermentation process for producing ethanol from whole corn. The process comprises fractionating the whole corn and separating the fractionated corn into a low oil fraction and a high oil fraction, the low oil fraction comprising starch. A slurry comprising water and the low oil fraction is formed that is then liquified to form a mash. A fermentation medium is formed comprising the mash, a source of added free amino nitrogen, yeast and backset, wherein the added free amino nitrogen content in the fermentation medium is at least about 1.2 milligrams of free amino nitrogen per gram of starch in the mash and the backset constitutes at least about 25 percent by volume of the fermentation medium. The fermentation medium is saccharified and fermented to produce a crude fermentation composition comprising ethanol that is then recovered.

Another aspect of the present invention is a fermentation process for producing ethanol from whole corn, the process comprising fractionating the corn into a low oil fraction and a high oil fraction, and separating the fractions with the low oil fraction comprising, on an anhydrous basis, less than about 3 percent by weight total oil, at least about 72 percent by weight starch, from about 5 percent by weight to about 11 percent by weight total protein, and less than about 20 percent by weight non-fermentables. A slurry comprising water and the low oil fraction is formed that is then liquified to form a mash. A fermentation medium is formed comprising the mash, an added source of free amino nitrogen and yeast, wherein the added free amino nitrogen content is at least about 1.2 milligrams of nitrogen per gram of starch in the fermentation medium. The fermentation medium is saccharified and fermented to produce a crude fermentation composition comprising ethanol that is then recovered.

The present invention is further directed to a distillers dried grain with solubles composition prepared from a fermentation process comprising fractionating and separating corn into a low oil, starch-containing fraction and a high oil fraction. A slurry comprising water and the low oil fraction is formed that is then liquified to form a mash. A fermentation medium comprising the mash and yeast is formed that is saccharified and fermented to produce a crude fermentation composition comprising crude dried distillers grain with solubles that is recovered. The DDGS comprises from about 7 to about 9 percent by weight total oil and greater than 35 weight percent total protein. The DDGS yield, calculated as the ratio of the weight of dried distillers grain with solubles produced, on an anhydrous basis, to weight of starch in the mash is less than 0.25.

Other objects and features will be in part apparent and in part pointed out hereinafter.

### BRIEF DESCRIPTION OF THE DRAWINGS

FIG. 1 is a schematic flow chart for the process of the present invention for the preparation of ethanol and distillers dried grain with solubles (DDGS).

FIG. 2 is a schematic flow chart for the process of the present invention for the preparation of ethanol and distillers dried grain (DDG).

### DESCRIPTION OF THE PREFERRED EMBODIMENTS

As noted, the present invention is directed to improved fermentation processes for the preparation of ethanol from a corn fraction having relatively high starch and low oil and low non-fermentable content, termed a low oil fraction (“LOF”); advantageously such processes are capable of providing improved ethanol production rate, improved yield from starch, improved ethanol yield per unit volume of fermenter capacity and ethanol having reduced fusel oil content. Also provided are improved processes for the preparation of distillers dried grain with solubles having low oil, low acid detergent fiber and high protein content.

It has been discovered that by the selection, combination and control of one or more process variables including, but not limited to, LOF composition, aeration, starch and free amino nitrogen (“FAN”) concentration, yeast strain, and yeast nutrients and micronutrients and their relative concentrations, industrial scale ethanol productivity rates in grams per liter per hour and ethanol yield based on starch can be achieved that could not be previously economically achieved. In one embodiment of the present invention, for example, from about 2.2 to about 3.9 grams of ethanol per liter per hour of fermentation composition and at a yield of from about 0.5 to about 0.56 grams of ethanol per gram of starch is obtained in from about 40 to about 55 hours on an industrial scale (e.g., fermentation volumes of at least 250,000 liters). In another measure, the process of the present invention achieves a starch conversion of 91%, 92%, 93%, 94% or 95%. By way of further example, the process of the present invention is capable of achieving industrial scale fermentation times of about 40 hours at an ethanol production rate of up to about 3.9 grams of ethanol per liter per hour and at an ethanol concentration up to about 20 percent by volume.

In another measurement of ethanol production rate, the low non-fermentable content of the LOF of the present invention provides an increase of about 1%, 2% or even 3% effective fermentation capacity on a unit volume basis because the reduced amount of non-fermentable material frees up fermentation volume that would otherwise be occupied by the non-fermentable material that is present in prior art fermentation processes. Greater effective fermentation volume directly results in an ethanol throughput increase of 1%, 2% or even 3% for the fermentation process of the present invention. The net result is production of from about 3 to about 4.1 grams of ethanol per liter of usable, or occupied, fermentation volume per hour of fermentation (“g EtOH/fermenter L/hr”) can be achieved by the process of the present invention. For example, about 3, 3.1, 3.2, 3.3, 3.4, 3.5, 3.6, 3.7, 3.8, 3.9, 4 or 4.1 g EtOH/fermenter L/hr.

In one preferred embodiment of the present invention, such ethanol production rates can be achieved using a fermentation medium comprising yeast, added free amino nitrogen, backset, and LOF containing, on an anhydrous basis, at least about 70 percent by weight starch, less than about 3 percent by weight oil, about 5 to about 11 percent protein and less than about 20 percent by weight non-fermentables.

The process of the present invention is generally directed to LOF liquifaction, yeast propagation in a mash comprising liquified LOF and optionally a source of added FAN, generation of a crude fermentation composition by saccharifying and fermenting a mash comprising liquified LOF, propagated yeast, and optionally a source of added FAN and backset, and isolation of ethanol and DDGS or DDG from the crude fermentation composition.

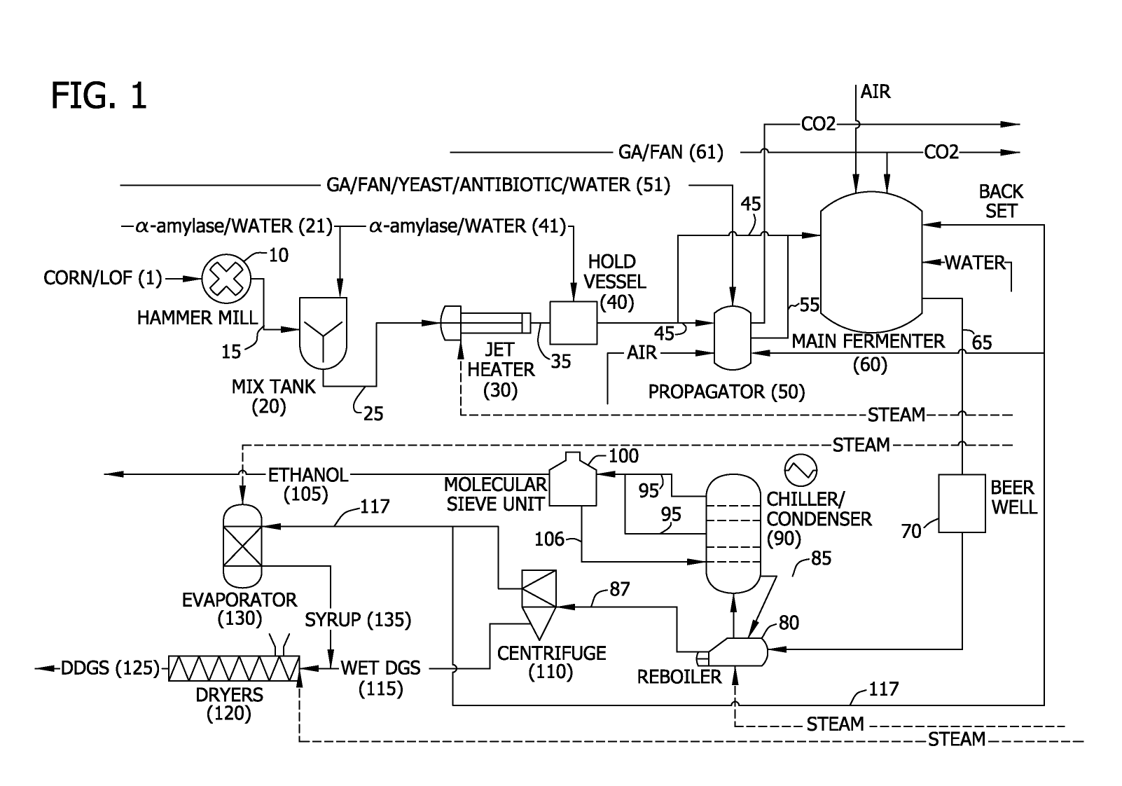

More particularly, in reference to FIG. 1, the process is generally directed to: LOF (1) coarse milling (10) to generate milled LOF (15); combining the milled LOF (15) with α-amylase and water (21) in a mix tank (20) in a pre-liquifaction step to form pre-liquified LOF (25); feeding the pre-liquified LOF (25) to a jet heater (30) for heat liquifaction to generate heat-liquified LOF (35); combining the heat-liquified LOF (35) with α-amylase and water (41) in a holding vessel (40) in a digest step to generate liquified LOF (45); combining a portion of the liquified LOF (45) with glucoamylase (“GA”), a FAN source, yeast, antibiotic and water (51) in a propagation tank (50) to generate pitching yeast (55); combining a portion of the liquified LOF (45) with the pitching yeast (55) and GA, a FAN source and water (61) in a fermenter (60) to generate a fermentation composition (65); sending the fermentation composition to a beer well (70) then to a reboiler (80) for recovery of crude ethanol (95) from the overhead stream (85) in a condenser (90); sending the crude ethanol (95) to a molecular sieve unit (100) for separation of ethanol (105) from byproducts (106); sending the reboiler bottoms (87) to a centrifuge (110) where wet distiller grain solids (“DGS”) (115) and centrifugate (117) are separated (all or a portion of the centrifugate (117) can optionally be recycled to the mix tank (20), propagation tank (50) and/or fermenter (60) as backset); feeding the centrifugate (117) to an evaporator (130) where it is concentrated to produce syrup (135); combining the syrup (135) with the wet DGS (115) and sending the combination to a dryer (120) in which DDGS (125) is prepared.

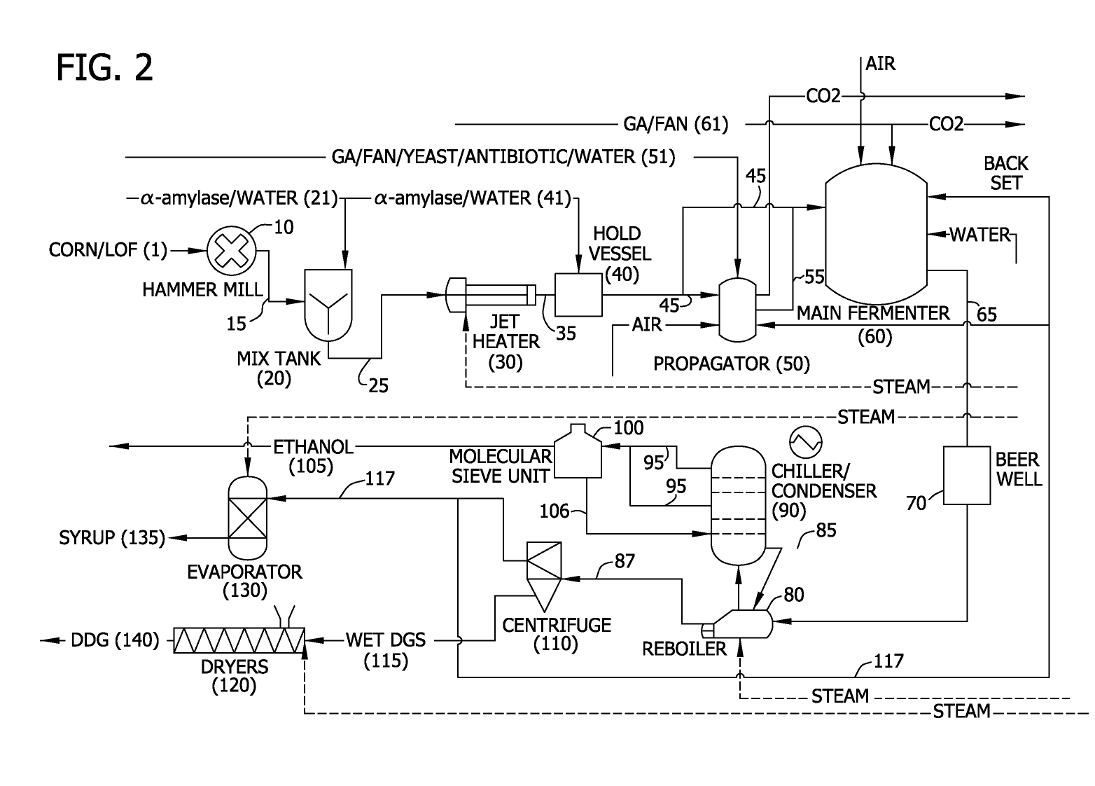

In one alternative embodiment, depicted in FIG. 2, wet DGS (115) is dried in the absence of syrup (135) to generate dried distillers grain (“DDG”) (140).

### LOF

Typical starting material for the preparation of LOF for fermentation is whole kernel corn seed or grain harvested from any of a wide variety of corn plants. Suitable corn types include, for example, conventional corn (e.g., yellow number 2); flint corn; popcorn; flour corn; dent corn; sweet corn; hybrids; inbreds; transgenic or genetically modified plants selected from highly fermentable, high oil, high lysine, hard endosperm, nutritional density, high protein, high starch, waxy corn and white corn; or combinations thereof.

In one embodiment, the starting corn grain is highly fermentable corn. As compared to yellow number 2 corn, highly fermentable corn possesses greater total starch content and greater starch availability as characterized by increased extractability and conversion to ethanol in a fermentation process. It is known that highly fermentable corn increases ethanol yield in fermentation processes by about 2% to about 4% as compared to commodity corn, such as yellow number 2, similarly processed. Highly fermentable corn is marketed, for example, by Monsanto (St. Louis, Mo., USA) as Processor Preferred®, by Syngenta as Extra Edge®, and by Pioneer as High Total Fermentable®.

In another embodiment, the starting corn grain is a high oil corn comprising, on a dry matter (anhydrous) basis, at least about 6 wt % oil. High oil corn is commercially available, for example, from Cargill Inc. (Minneapolis, Minn., USA), Monsanto, Pfister Hybrid Corn Co. (El Paso, Ill., USA), Wyffels Hybrids Inc. (Geneseo, Ill., USA), Galilee Seeds Research and Development (Rosh Pina, Israel) and DuPont Specialty Grains (Johnston, Iowa, USA). Other suitable high oil corn includes the corn populations known as Illinois High Oil (IHO) and Alexander High Oil (Alexo), samples of which are available from or through the University of Illinois Maize Genetics Cooperation Stock Center (Urbana, Ill., USA). Examples of high oil corn include DuPont OPTIMUM™; AgriGold hybrids A6453TC and A6490; Monsanto DK621TC; Asgrow hybrids 748TC and RX730TC; Golden Harvest H9257; Burrus 560 TC3; Croplan hybrids 6607ED and 6611ED; TopCross® blends available from Pfister as hybrids SK2550-19, SK2650-19, SK2652-19, SK2680-19, SK3001-19 and SK3049-19; and Pioneer 34B25. Methods for developing corn inbreds, hybrids, transgenic species and populations that generate corn plants producing grain having elevated oil concentrations are known and described in the art. See, for example, Lambert, Specialty Corn, CRC Press Inc., Boca Raton, Fla., USA, pages 123-145 (1994) and United States Patent Application Publication No. 2003/0182697. Although sometimes less preferred, conventional yellow corn, having an oil content of, for example, about 3 wt % to about 6 wt % may also be used.

In another embodiment, the starting corn grain is a high lysine corn typically containing at least about 3000 parts per million (“ppm”), 3500 ppm, 4,000 ppm, 5,000 ppm, 6,000 ppm, 7,000 ppm or even 8,000 ppm total lysine. One example of high lysine corn is Renessen MAVERA™ high value corn with lysine.

In another embodiment, (a) corn varieties having traits such as highly fermentable, hard endosperm, waxy, white, nutritionally dense, high protein or high oil, (b) corn varieties having combinations of traits selected from two or more of highly fermentable, high oil, hard endosperm, waxy, white, nutritionally dense, high protein and high starch, or (c) a mixture of two or more corn varieties having traits selected from highly fermentable, high oil, high lysine, hard endosperm, waxy, white, nutritionally dense, high protein and/or high starch can be fermented according to the process of the present invention. Hard endosperm varieties include, for example, AgriGold hybrids A6427 and A6490, QTIC QC9664, LG Seeds C7847, Pioneer hybrids 34K77 and 33P66, Burrus 442, LG Seed LG2587, Horizon Genetics 7460CL, and Trisler T5313. Waxy varieties include, for example, Novartis N4342, Pioneer hybrids 34H98 and 33A63, and DeKalb 624WX. White varieties include, for example, Pioneer hybrids 34P93 and 32Y52, Asgrow 776W, Trisler T4214, and AgriGold 6530. Nutritionally dense varieties include, for example, Adler 4100, Diener 105, Lewis ND5000, Growmark 6581ND, Beck EX1924, Bird hybrids ND70 and ND74, Croplan hybrids TR1049ND, E557, E560 and E565, Exseed Nutridense® hybrids 5109ND and 5110ND, Mycogen hybrids 2654 and 2655, Seed Consultants 11N00, Seedway 618HOC, and Wellman hybrids WIN 109 and WIN 111. An example of a high protein variety is Diener 108S and an example of a high starch variety is Novartis N59-Q9.

In one embodiment, LOF is prepared from whole corn by a process in which the whole corn kernels are conveyed to a fractionating apparatus, such as a Buhler-L or Buhler-Mapparatus (Buhler GmbH, Germany), wherein the kernels are contacted with an abrasive device to separate a portion of the hull and the germ component (i.e., the portion of the corn material containing the corn germ, fractions of corn germ, components of germ, and oil bodies) from the remainder of the corn material, generally comprising the endosperm. Where a screen is used as the abrasive device, a portion of the hull and germ component pass through the screen(s) and form a high oil fraction (“HOF”) that is typically extracted to generate corn oil and a solvent extracted high oil fraction (“SEHOF”). SEHOF is characterized by a high germ content and low oil content. Germ is rich in crude protein and can be a useful nutrient and assimilable nitrogen source for yeast in fermentation operations. SEHOF typically comprises, on an anhydrous basis, less than about 1.7 wt % oil, a protein content of from about 9 wt % to about 25 wt %, a total lysine content of between about 0.4 wt % and about 0.6 wt %, a starch content of from about 30 wt % to about 70 wt %, and a neutral detergent fiber (“NDF”) content of from about 12 wt % to about 24 wt %. The material left on the screen(s) comprises the LOF and some germ component. The ratio of LOF to HOF is preferably about 50:50, about 55:45, about 60:40, about 65:35, about 70:30, about 75:25, about 80:20, about 85:15 or even about 90:10. The ratio range is preferably about 50:50 to about 90:10, about 60:40 to about 90:10, about 70:30 to about 90:10, about 75:25 to about 90:10, or even about 80:20 to about 90:10.

In general, the resulting LOF will typically have a relatively low total non-fermentable content, low oil content (a non-fermentable component) and high starch content. For example, on an anhydrous basis, the LOF will typically have an oil concentration of less than about 6 wt %, for example, less than 5%, 4%, 3%, 2%, 1% or even 0.5% by weight on an anhydrous basis. In addition, the resulting LOF will typically contain at least 65 wt % starch. In one embodiment, for example, the LOF preferably contains at least 70 wt % starch. In some embodiments, the starch content will be even greater, constituting at least 72%, 75%, 80%, 85% or even 90% by weight of the LOF, on an anhydrous basis.

Other components of the resulting LOF include non-fermentable materials such as oil (described above), fiber, protein and ash. Crude protein content is preferably about 5 wt % to 11 wt % on an anhydrous basis. Acid detergent fiber (“ADF”) content is preferably less than about 7 wt %, for example, 5 wt %, 3 wt %, 2 wt % or even 1 wt % on an anhydrous basis. Neutral detergent fiber (“NDF”) content is preferably less than about 12 wt %, for example, 10 wt %, 8 wt %, 6 wt %, 4 wt % or even 3 wt % on an anhydrous basis. Ash content is preferably less than about 1.2 wt %, for example, 1 wt %, 0.8 wt %, 0.6 wt %, 0.4 wt % or even 0.3 wt % on an anhydrous basis. Total non-fermentable content is preferably less than about 30 wt %, 25 wt % 20 wt %, 15 wt % or even 10 wt % on an anhydrous basis. Non-fermentable material occupies space in the fermentation equipment that would otherwise be available for fermentation thereby reducing the effective fermentation capacity. The low non-fermentable content of the LOF of the present invention advantageously results in an increase of about 1%, 2% or even 3% in effective fermentation capacity on a unit volume basis. That effective volume increase provides a corresponding (1) ethanol yield increase on a fermentation volume basis and (2) throughput increase.

### Milling

In one embodiment, LOF is milled to produce a coarse ground material. Suitable mills include ball mills, hammer mills and roller mills. In the case of a hammer mill, a screen size opening of from about 2 mm to about 5 mm, for example, about 3 mm (⅛ inch) is typically used. Typical LOF particle size is generally characterized as less than about 35%, 40%, 45%, 50%, 60%, 70%, 80% or even 90% of the LOF particles pass through a 0.5 mm to a 1 mm sieve opening.

In another embodiment, LOF is milled through a screen size opening of from about 0.1 mm to about 2 mm to produce LOF particle size generally characterized as having greater than about 50%, 60,%, 70%, 80% or even 90% of the LOF particles pass through a sieve opening of from about 0.1 mm to about 0.5 mm.

### Mixing and Primary Liquifaction

In a primary liquifaction step, LOF or milled LOF is combined with water and an α-amylase enzyme in a heated mixing tank to form a heated suspension. The α-amylase enzyme and heat liquifies at least a portion of the starch contained in the LOF to form pre-liquified LOF comprising oligosaccharides.

In one embodiment, at least 25%, for example 25%, 50%, 75%, 90%, 95% or even 100% of the water added to the mix tank can be replaced with backset (117) (centrifugate) recycled from the centrifuge (110). Experimental evidence to date indicates that increasing fermentation production rates are positively correlated with backset addition. Under one theory, and without being bound to any particular theory, it is believed that backset provides essential yeast nutrients and micronutrients, and serves as a pH buffer. In one backset addition option, less than complete backset recycle is used, for example, from about 20% to about 75% backset addition to the mix tank, in order to purge a portion of the fermentation impurities and inhibiters from the process.

In one embodiment, a LOF suspension having a dry solid (“DS”) weight percent content preferably from about 20% to about 45%, more preferably from about 25% to about 35%, for example 26%, 27%, 28%, 29%, 30%, 31%, 32%, 33%, 34% or 35% is prepared. A pH of about 5 to 6 is preferred. If necessary, the pH can be adjusted to that range with a mineral acid such as sulfuric acid, hydrochloric acid or nitric acid, or with a base such as sodium hydroxide or ammonia (ammonium hydroxide). It has been discovered that LOF typically forms slurries having a pH of about 5 to 6 thereby obviating the need for pH adjustment. Under one theory, and without being bound to any particular theory, it is believed that LOF is relatively unbuffered as compared to standard corn. Prior art standard corn mashes are typically acidic and require pH adjustment to a range of 5 to 6 with a base, such as ammonium hydroxide. Experimental evidence to date indicates that LOF mashes require less base resulting in a reduction in base usage of from about 10% to about 80%. Minimizing pH adjustment reduces the amount of salts generated during fermentation which are known to precipitate from solution during evaporation resulting in evaporator fouling and reduced evaporation efficiency. The mixing temperature is generally maintained from about 30° C. to about 85° C., for example, about 80° C., about 75° C., about 70° C., about 65° C., about 60° C., about 55° C., about 50° C., about 45° C., about 40° C., about 35° C. or even about 30° C.

Typical α-amylase enzymes used in the practice of the present invention convert starch to fermentable sugars and are of fungal or bacterial origin. In general, α-amylases effect random cleavage α-(1-4) glucosidic linkages in starch thereby hydrolyzing the starch to generate maltodextrins (dextrins). Examples of typical bacterial and fungal amylases include, for example, enzymes derived from B. licheniformis, B. amyloliquefaciens, B. stearothermophilus, Aspergillus oryzae and Aspergillus niger.

In one embodiment, the α-amylase is an acid α-amylase having enzymatic activity at a pH in the range of about 3 to about 7, about 3.5 to 6 or even from about 4 to 5. Examples of commercial acid α-amylases suitable for use in the present invention include TERMAMYL™ SC, LIQUOZYME™ SC and SAN™ SUPER (all available from Novozymes A/S, Denmark); and DEX-LO™, SPEZYME™ FRED, SPEZYME™ AA, and SPEZYME™ DELTA AA (all available from Genencor).

In another embodiment, the α-amylase is a thermostable acid α-amylase having enzymatic activity at a temperature of up to about 90° C. and at a pH in the range of about 3 to about 6, or about 3.5 to about 6. Examples of commercial thermostable acid α-amylases suitable for use in the present invention include FUNGAMYL® (available from Novozymes A/S) and Clarase™ (available from Genencor Int., USA).

It has been discovered that fermentation of ground corn, as compared to LOF, typically requires greater amounts of amylase enzymes to liquify the starch material. Without being bound to any particular theory, it is believed that amylase enzyme activity is reduced by non-fermentable loading and the presence of Mg²⁺ and other heavy metals that are typically found in the corn germ component. Generally, excess Ca²⁺ (about 10 to 100 ppm) is preferably present to stabilize the amylase enzyme during liquifaction. Advantageously, LOF contains low non-fermentable and germ content and, as compared to whole corn, low heavy metal concentration therefore reducing α-amylase requirements.

The quantity of α-amylase useful for liquifaction is known in the art. In general, α-amylase activity is preferably high enough to result in a primary liquifaction suspension having a dextrose equivalent (“DE”) concentration of about 5, about 6, about 7, about 8, about 9 or even about 10. In the case of bacterial acid α-amylase, enzyme activity is preferably present in an amount of about 0.05 to about 100 acid α-amylase units per gram of DS (AAU/g), from about 0.1 to about 50 AAU/g of DS, more preferably from 0.5 about to about 10 AAU/g of DS. In the case of fungal acid α-amylase, enzyme activity is preferably present in an amount of from about 0.01 to about 10 acid fungal α-amylase units per gram of DS (AFAU/g), more preferably from about 0.1 to about 5 AFAU/g.

### Heat Liquifaction

In one heat liquifaction embodiment, a liquified LOF suspension comprising oligosaccharides is prepared from the primary liquified LOF suspension in two steps. In the first step, the primary liquified LOF suspension is passed through a jet heater which sparges steam by direct injection throughout the suspension resulting in a heat-liquified LOF suspension having a temperature of 70° C., 75° C., 80° C., 85° C., 90° C., 91° C., 92° C., 93° C., 94° C., 95° C., 96° C., 97° C., 98° C., 99° C., 100° C., 101° C., 102° C. or 103° C., or ranges thereof. The residence time in the heater is 1 minute, 5 minutes, 10 minutes, 15 minutes, 20 minutes, 25 minutes or even 30 minutes. It is believed that a portion of the starch is liquified by a combination of heat and the steam-induced shear and mechanical forces. It will be appreciated that the temperature, pressure, and residence time are interdependent so that the modifications in any of those variables may be made in order to accommodate the heat liquifaction process into the fermentation process.

In the second step, the heat-liquified LOF suspension is digested to produce a finished liquified LOF suspension having a DE of about 8, about 9, about 10, about 11, about 12, about 13, about 14, about 15, or ranges thereof, for example from about 8 to about 15, or from about 10 to about 15. In one embodiment, the liquifaction process is continuous and the α-amylase is added to the heat-liquified LOF suspension at it exits the jet heater, and the holding vessel is a plug flow vessel. The heat-liquified LOF suspension passes from the jet heater into one or more holding vessels, such as a horizontal tank, having sufficient volume to provide a residence time of from about 30 minutes to about 2 hours at a temperature of from about 55° C. to about 90° C. In one embodiment, during the digestion step, the LOF suspension is treated with additional α-amylase in a second enzyme stage, alone or in combination with one or more other enzymes such as glucoamylase, β-amylase, pulluanase, glucose isomerase or protease, sequentially or in combination. In another embodiment, the liquifaction process is semi-continuous and the heat-liquified LOF is fed from the jet heater to a hold tank for batch-wise digestion and/or second stage enzymatic treatment. In yet another embodiment, the pre-liquified LOF suspension is heated and digested (optionally including a second enzyme stage) in a batch process in one vessel or a series of vessels. If thermostable α-amylase is used alone in the second enzyme stage, the temperature will preferably range from about 80° C. to about 110° C., for example about 85° C. If other enzymes are present such as glucoamylase, the temperature is preferably somewhat lower, for example, 55° C. to 75° C. Typical α-amylase enzyme dosages are described above.

### Yeast Propagation

In one embodiment, the yeast can be grown and conditioned in a propagation step by incubating it in a fermentation medium comprising the liquified LOF suspension in a propagation tank to produce pitching yeast. The yeast source can be dried yeast or a yeast inoculate suspension. Any of a variety of yeasts can be employed as the yeast in the present process. Typical yeasts include any of a variety of commercially available yeasts, such as commercial strains of Saccharomyces cerevisiae. Typical strains include ETHANOL RED (available from Red Star/Lesaffre, USA); BioFerm AFT, HP and XR (available from North American Bioproducts); FALI (available from Fleischmann's Yeast, a division of Burns Philp Food Inc., USA); SUPERSTART (available from Lallemand); GERT STRAND (available from Gert Strand AB, Sweden); FERMIOL (available from DSM Specialties); and Thermosac (available from Alltech).

Incubation is typically performed at an initial yeast concentration of about 0.1 to about 5, about 0.5 to about 3, or even from about 0.5 to about 1 grams of yeast per liter of the fermentation medium. On a yeast count basis, in yeast cells per liter, the initial concentration is about 2×10⁹ to about 2×10¹¹, 1×10¹⁰ to about 1×10¹¹ or even 1×10¹⁰ to about 5×10¹⁰.

Added FAN can be optionally included in the fermentation medium to supplement FAN present in the LOF and/or corn. Yeast cells require nitrogen atoms for the formation of, for example, proteins, enzymes, co-enzymes and nucleic acids that are needed for cell propagation and metabolism. The major sources of nitrogen for yeasts are collectively termed FAN and include the contribution of nitrogen from many sources including, but not limited to: organic compounds such as urea; inorganic compounds such as ammonium sulfate and ammonia (ammonium hydroxide); amino acids; and α-amino nitrogen groups of peptides and proteins. FAN is typically expressed as milligrams of nitrogen per liter (mg N/L), but can be normalized based on starch content and be alternatively expressed in milligrams of nitrogen per gram of starch (mg N/g starch). FAN can be measured using a variety of analytical methods. In one method, FAN can be measured using a spectrophotometric method (Perkin Elmer LS50B) which displays and measures a color reaction between ninhydrin and the nitrogen present in the sample (International Method of the Technical Committee and Editorial Committee of the American Society of Brewing Chemists (1992)). The amount of absorbance is directly related to the amount of FAN present. In another method, FAN can be determined by the AOAC method (15^{th} ED. 1990. pg. 735).

Typical added FAN sources include urea, ammonium sulfate and ammonia (ammonium hydroxide). Added FAN content is preferably from about 1.2 to about 6 mg N/g starch, for example 1.2, 2.4, 3.6, 4.8 or 6 mg N/g starch. In the case of urea, it is preferred to add from about 2.4 to about 12 mg urea per gram of starch, for example, 2.4, 4.8, 7.2, 9.6 or 12 mg urea per gram of starch.

In another embodiment, as described herein, backset can be added to the fermentation medium. When backset is added, at least 25%, for example 25%, 50%, 75%, 90%, 95% or even 100% of any water added water to the propagator (including water present in the liquified LOF suspension) can be replaced with backset in order to increase the nutritive content of the fermentation medium. In one embodiment, less than 100% of the water added to the propagator is replaced with backset to allow for purging of backset impurities.

Bactericides can also optionally be added to the fermentation medium. Examples of typical bactericides include virginiamycin, nisin, erythromycin, oleandomycin, flavomycin, penicillin G. In the case of virginiamycin, a concentration of from about 1 ppm to about 10 ppm is preferred.

Yeast foods that supply, for example, vitamins (such a B vitamins and biotin), minerals (such as from salts of magnesium and zinc) and micronutrients and nutrients can also be added to the fermentation medium. Yeast foods can include autolyzed yeast and plant extracts and are typically added to a concentration of from about 0.01 to about 1 g/L, for example from about 0.05 to about 0.5 g/L.

Enzymes can also be added to the fermentation medium, with examples including proteases, phytases, cellulases, hemicellulases, xylanases, and/or exo- and endo-glucanases.

The oligosaccharides resulting from the liquifaction process are converted to smaller polysaccharides and eventually to monosaccharides, such as glucose, by hydrolysis in a saccharification step. The hydrolysis is preferably preformed enzymatically by addition of a glucoamylase, alone or in combination with other enzymes, such as an alpha-glucosidase, a protease and/or an acid alpha-amylase. Glucoamylase is an exoenzyme since it attacks the ends of the starch molecules and oligosaccharides. The enzyme hydrolyzes both 1,4 and 1,6 linkages, so nearly complete hydrolysis of the starch can be achieved. In one embodiment, the glucoamylase enzyme is added prior to the yeast in a pre-saccharification step, lasting for about 15 minutes to about 2 hours, for example from about 30 minutes to about 60 minutes. In another embodiment, yeast and glucoamylase are added to the fermentation medium at a closely spaced time interval. In either embodiment, when glucoamylase enzyme and yeast are both present, fermentation and saccharification predominantly occurs simultaneously.

Typical glucoamylase enzymes for use according to the present invention may be derived from any suitable source, e.g., from a microorganism or a plant. Preferred glucoamylases are of fungal or bacterial origin and are selected from the group consisting of Aspergillus glucoamylases, in particular A. niger G1, A. niger G2, A. awamori, A. oryzae, or variants or fragments thereof. Other Aspergillus variants include variants to enhance the thermal stability. Still other glucoamylases include Talaromyces glucoamylases such as those derived from Talaromyces emersonii, Talaromyces leycettanus, Talaromyces duponti, Talaromyces thermophilus. Bacterial glucoamylases contemplated include glucoamylases from the genus Clostridium, in particular C. thermoamylolyticum, and C. thermohydrosulfuricum. Commercially available compositions comprising glucoamylase include: AMG 200L, AMG 300 L, AMG E, SAN™ SUPER, SAN™ EXTRA L, SPIRIZYME™ PLUS, SPIRIZYME™ FUEL, SPIRIZYME™ FG and SPIRIZYME™ E (all available from Novozymes); OPTIDEX™ 300 and Distillase L-400 (available from Genencor Int.); AMIGASE™ and AMIGASE™ PLUS (available from DSM); G-ZYME™ G900, GZYME™ 480 Ethanol and G990 ZR (all available from Genencor Int.). Glucoamylases may be added in an amount of from about 0.02 to about 20 glucoamylase units per gram DS (“AGU/g”), from about 0.1 to about 10 AGU/g DS, or even from about 1 to about 5 AGU/g DS.

Depending upon the conditions, pitching yeast propagation incubation time can be from about 6 hours to about 24 hours, from about 8 hours to about 16 hours, from about 8 hours to about 12 hours, or even from about 10 hours to about 12 hours. A propagation pH of 3.5 to about 6, from about 3.5 to about 5, or even from about 3.8 to about 5 is preferred. A propagation temperature of from about 30° C. to about 36° C., from about 31° C. to about 35° C. or even from about 32° C. to about 34° C. is preferred. In one embodiment, the propagation fermentation medium is aerated resulting in a dissolved oxygen concentration of about 5 ppm, 6 ppm, 7 ppm, 8 ppm, 9 ppm or even 10 ppm. The finished pitching yeast propagation suspension generally contains a yeast concentration of from about 1×10⁸ to about 1×10⁹ cells/mL, for example, about 5×10⁸ cells/mL is preferred.

### Fermentation

In the fermentation step, a fermentation medium comprising pitching yeast and liquified LOF suspension is formed in a fermentation vessel, the medium having a volume ratio of LOF suspension to pitching yeast of from about 10:1 to about 100:1, from about 25:1 to about 75:1, or even from about 40:1 to about 60:1, for example, about 50:1. A fermentation vessel volume of at least 1,000 liters, 10,000 liters, 50,000 liters, 100,000 liters, 500,000 liters, 1,000,000 liters, 2,000,000 liters or even 3,000,000 liters is preferred. The fermentation medium is then fermented to produce a crude fermentation composition.

In an optional embodiment, dried yeast such as ETHANOL RED, BioFerm AFT, BioFerm HP, Bioferm XR, FALI, SUPERSTART, GERT STRAND, FERMIOL or Thermosac can be added directly to the fermenter without first propagating the yeast. The dried yeast addition amount is sufficient to preferably supply from about 5×10⁷ to 5×10⁸ cells per mL in the liquified LOF suspension.

After yeast addition, a fermentation mixture starch concentration of at least about 200, 210, 220, 230, 240, 250, 260, 270, 280, 290 or even 300 grams per liter is preferred. In another measure, a total dissolved solids (“TDS”) content of at least 25%, 26%, 27%, 28%, 29%, 30%, 31%, 32%, 33%, 34% or even 35% is preferred. In yet another measure, a DE value of at least 6, 7, 8, 9, 10, 11, 12, 13, 14 or even 15 is preferred. If the DE is too high some of the free dextrose produced in liquifaction can convert to non-fermentable sugars. Conversely, if the DE is too low viscous suspensions can result and the ethanol production rate can be reduced.

In one yeast addition embodiment, the yeast pitch addition to the fermenter can be done during the fermenter fill cycle. In another yeast addition embodiment, the yeast pitch can be split by adding a first portion during the fermenter fill cycle and then adding the remainder of the yeast in one or more subsequent additions later in the fermentation cycle at elapsed fermentation times selected to minimize yeast stress and maximize ethanol yield (in grams of ethanol per liter and/or in grams of ethanol per gram of starch) and ethanol production (in grams of ethanol per liter per hour). For example, about one half of the yeast can be added during the beginning of the fermenter fill cycle and the remainder can be added from about 4 hours to about 12 hours later, or from about 6 to 10 hours later. In yet another embodiment, a first portion of the yeast can be added during the fermenter fill cycle and the remainder can be added continuously over a subsequent 4 hour, 5 hour, 6 hour, 7 hour, 8 hour, 9 hour, 10 hour, 11 hour or 12 hour time period. The continuous addition rate can be controlled as an output from a continuous control loop having one or more fermentation variables as inputs, such as, for example, glucose concentration, ethanol concentration, impurity concentration, density, glucoamylase addition rate, FAN addition rate, aeration rate, and/or carbon dioxide off-gassing rate. Various regulatory control schemes can be utilized including, for example, closed control loops, feed forward control, feed forward control with feedback trim, ratio control, cascaded control, and cascaded control in combination with feed forward control.

The oligosaccharides resulting from the liquifaction process are converted to smaller polysaccharides and eventually to monosaccharides, such as glucose, by hydrolysis in a saccharification step. The hydrolysis is preferably preformed enzymatically by addition of a glucoamylase, alone or in combination with other enzymes, such as an alpha-glucosidase, a protease and/or an acid alpha-amylase. Glucoamylases may be added in an amount of from about 0.02 to about 20 glucoamylase units per gram DS (“AGU/g”), from about 0.1 to about 10 AGU/g DS, or even from about 1 to about 5 AGU/g DS. In one embodiment, the glucoamylase enzyme is added prior to the yeast in a pre-saccharification step, lasting for about 15 minutes to about 2 hours, for example from about 30 minutes to about 60 minutes. In another embodiment, yeast and glucoamylase are added to the fermentation medium at a closely spaced time interval. For example, glucoamylase and yeast can be added concurrently according to the yeast addition embodiments described above. In any embodiment, when glucoamylase enzyme and yeast are both present, fermentation and saccharification predominantly occurs simultaneously.

In one preferred embodiment, as described above in the yeast propagation step, FAN can be added to the fermentation medium. Typical FAN sources include ammonium sulfate and urea. Added FAN concentration is preferably from about 1.2 to about 6 mg N/g starch, for example about 1.2, 2.4, 3.6, 4.8 or even 6 mg N/g starch. In the case of urea, it is preferred to add from about 2.4 to about 12 mg urea per gram of starch, for example, about 2.4, 4.8, 7.2, 9.6 or even 12 mg urea per gram of starch. In the case of urea, experimental evidence to date indicates that an addition rate of from about 2.4 to about 6 mg urea per gram of starch (about 1.2 to about 3 mg N/g starch) provides about a 5%, 10%, 15%, 20% even 25% decrease in fermentation time to completion. In a similar measure, about a 10% increase in ethanol production rate at completion was found. Fermentation completion times of 42 hours at 3.6 grams of urea per gram of starch were observed as compared to about 56.5 hours for an added urea concentration of about 1.2 mg per gram of starch. In another measure, fermentation rate was observed to be about 40% to about 50% greater at 42 hours for 3.6 mg added urea per gram of starch as compared to 1.2 mg added urea per gram of starch. Complete fermentations are generally defined as the point at which the ethanol concentration reaches a maximum and/or the carbohydrate concentration in the fermentation mixture is less than about 3,000 ppm to about 10,000 ppm.

In any embodiment, FAN addition can occur before, after and/or simultaneously with the addition of other fermentation components such as liquified LOF suspension, yeast, glucoamylase and other additives. The addition can be done at the start of the fermentation or can optionally be done in two or more additions during the fermentation according to an addition schedule. Optionally, the FAN and/or glucoamylase can be added continuously during a portion of the fermentation according to an addition schedule designed to maximize ethanol yield and production rates. For example, ethanol production rates are typically rapid during the first third of the fermentation cycle (about the first 12, 15, 18 or even 20 hours of the fermentation) and the rate declines as fermentable sugars are depleted and ethanol concentration rises. Fermentation essentially ceases when the ethanol concentration is sufficient to retard yeast activity and/or the fermentable sugar concentration drops to from about 3,000 ppm to about 10,000 ppm. In one embodiment, about half of the FAN and/or glucoamylase are added at the beginning of the fermentation and the remainder is added at about the 12 to 30 hour point in the fermentation, or even about the 15 to 25 hour point. In another embodiment, the FAN and/or glucoamylase are added in three or more additions over the first 30 hours of the fermentation. In still another embodiment, FAN and/or glucoamylase addition rates can be controlled as an output from a continuous control loop having one or more fermentation variables as inputs, such as, for example, glucose concentration, ethanol concentration, impurity concentration, yeast addition rate, density and/or carbon dioxide off-gassing rate. Various regulatory control schemes can be utilized including, for example, closed control loops, feed forward control, feed forward control with feedback trim, ratio control, cascaded control, and cascaded control in combination with feed forward control.

In another embodiment, as described herein, backset can be added to the fermentation medium. When backset is added, at least 25%, for example 25%, 50%, 75%, 90%, 95% or even 100% of any water added to the fermenter, including water contained in liquified mash and yeast pitch, can be replaced with backset in order to increase the nutritive content of the fermentation medium. Experimental evidence to date indicates that increasing fermentation production rates are positively correlated with backset addition. In particular, 25% water replacement with backset increases LOF fermentation rate by about 5% to about 10%, 50% water replacement increases LOF fermentation rate by about 10% to about 15%, and 100% water replacement increases LOF fermentation rate by about 15% to about 20%. Under one theory, and without being bound to any particular theory, it is believed that backset provides essential yeast nutrients and micronutrients, and serves as a pH buffer. It is believed that, over time, impurities can accumulate in the process under complete recycle conditions. Preferably, at least a portion of the backset (in the form of centrifugate (117)) is purged from the process. In one embodiment therefore, less than 100%, for example 90% or 95%, of the backset is recycled.

In one embodiment, the fermentation is aerated. Limited aeration during the early fermentation cycle, or about the first 20 hours, 15 hours, 14 hours, 13 hours, 12 hours, 11 hours, 10 hours, 9 hours, 8 hours, 7 hours, 6 hours, 5 hours or even 4 hours of the fermentation, increases yeast carbohydrate utilization thereby enabling fermentations to complete in shorter times and at elevated ethanol production rates (in grams of ethanol per liter per hour). It is believed, without being bound to any particular theory, that yeast require a limited amount of oxygen to synthesize certain compounds such as, for example, fatty acids and sterols that are required for yeast membrane synthesis. Development of robust cell membranes are a factor for growth and carbohydrate metabolism, particularly at high ethanol concentrations. In particular, an aeration rate of from about 0.005 to about 0.05 or from about 0.01 to about 0.03 standard liters per minute of air per liter of fermentation mixture is preferred. Because yeast rapidly utilize dissolved oxygen, appreciable dissolved oxygen is typically not attained in fermentation mixtures. However, in embodiments where mash is aerated prior to yeast addition, a total dissolved oxygen concentration of about 5 ppm to about 8 ppm is preferred. Experimental evidence to date indicates that aeration for the first about 5 hours to about 10 hours of the fermentation provides about a 10% decrease in fermentation time to completion. Aeration can be done directly by sparging air subsurface into the fermentation mixture or indirectly by introducing air into a fermentation mixture recycle stream.

In one embodiment, SEHOF is added to the fermentation process as a source of fermentable starch, FAN, micronutrients and lysine. SEHOF can be added to the mix tank (20) in the primary liquifaction step and/or to the fermenter (60). A ratio of SEHOF to LOF of 5:95, 10:90, 20:80, 30:70, 40:60 or 50:50, and ranges thereof, for example 5:95 to 40:60, is preferred.

As described above in the yeast propagation step, bactericides, yeast food, enzymes phytases, cellulases, hemicellulases, xylanases, and/or exo- and endo-glucanases can be optionally added to the fermenter before, after or simultaneously with yeast addition. Examples of typical bactericides include virginiamycin, nisin, erythromycin, oleandomycin, flavomycin, penicillin G. In the case of virginiamycin, a concentration of about 1 ppm to about 10 ppm is preferred. Yeast foods that supply, for example, vitamins (such a B vitamins and biotin), minerals (such as from salts of magnesium and zinc) and micronutrients and nutrients are preferred. Yeast foods can include autolyzed yeast and plant extracts and are typically added to a concentration of from about 0.01 to about 1 g/L, for example from about 0.05 to about 0.5 g/L.

In one embodiment, an acid protease enzyme can be added to the fermentation to generate short chain polypeptides from the protein fraction contained in the LOF and, if present, SEHOF. Protease addition is generally preferred when SEHOF is used as a FAN source. The short chain polypeptides can be used by the yeast for biological activities. Acid proteases suitable for the practice of the present invention include, for example, GC106 (available Genencor International) and AFP 2000 (available from Solvay Enzymes, Inc.). The amount of an acid protease is typically in the range of from about 0.01 to about 10 SAPU per gram of starch, from about 0.05 to about 5 SAPU per gram of starch, or even from about 0.1 to about 1 SAPU per gram of starch. As used herein “SAPU” refers a spectrophotometric acid protease unit, wherein 1 SAPU is the amount of protease enzyme activity that liberates one micromole of tyrosine per minute from a casein substrate under conditions of the assay. Experimental evidence to date indicates that protease addition increases LOF fermentation rate by about 5% to about 10%.

A fermentation pH of 3.5 to about 6, from about 3.5 to about 5, or even from about 3.8 to about 5 is preferred. It has been discovered that LOF naturally results in a fermentation pH of from about 3.5 to about 5.5 thereby reducing or eliminating the need for pH adjustment. If pH adjustment is required, minerals acids such as sulfuric acid, hydrochloric acid or nitric acid may be used or bases such as ammonia (ammonium hydroxide) or sodium hydroxide may be used. As previously described, reducing or eliminating pH adjustment reduces the amount of salts generated during fermentation.

A fermentation temperature of from about 30° C. to about 36° C., from about 31° C. to about 35° C. or even from about 32° C. to about 34° C. is preferred.

Based on experimental evidence to date, by the selection, combination and control of one or more process parameters including, but not limited to, LOF composition, yeast strain, yeast addition method, aeration rate, starch concentration, FAN concentration, metered and controlled FAN and/or glucoamylase addition, backset addition, SEHOF addition, yeast nutrients and micronutrients and their relative concentrations, and proteases, industrial scale fermentations having completion times of from about 32 hours to about 50 hours, for example, about 32, 36, 40, 44, 48 or hours (and ranges thereof), and ethanol concentrations of up to about 15, 15.5, 16, 16.5, 17, 17.5, 18, 18.5, 19, 19.5 or even 20 volume percent can be realized. Stated differently, ethanol concentrations of about 120 g/L, 130 g/L, 140 g/L, 150 g/L or even about 160 g/L, and ranges thereof, can be realized. In another measure, ethanol productivity (as measured in grams of ethanol per liter per hour at fermentation completion) of 2.1, 2.2, 2.3, 2.4, 2.5, 2.6, 2.7, 2.8, 2.9, 3, 3.1, 3.2, 3.3, 3.4, 3.5, 3.6, 3.7, 3.8 or even 3.9 can be realized. Further, fermentation compositions having reduced impurities can be prepared. Experimental evidence to date indicates that fermentation compositions can be prepared by the process of the present invention having a glycerol concentration of less than about 12 grams per liter (“g/L”), for example 11, 10, 9 or even 8.6 g/L; having a fusel oil concentration of about 0.5, 0.4, 0.3, 0.2 or even 0.1 g/L or less; having a weight ratio of ethanol to fusel oil of at least about 95:1, 100:1, 200:1, 300:1, 400:1, 500:1, 600:1, 700:1, 800:1, 900:1 or 1000:1; and having an acetic acid concentration of less than about 1 g/L, for example 0.8, 0.6, 0.4 or even 0.2 g/L.

Experimental evidence to date indicates that the process of the present invention provides a yield of ethanol based on starch of at least 0.5, 0.51, 0.52, 0.53, 0.54 or even 0.55 grams of ethanol per gram of starch. Those yields are higher than for ethanol prepared from flaked corn grits (about 0.49 grams ethanol per gram flaked grits). In another measure, starch conversion for LOF of greater than 90%, for example 91%, 92%, 93%, 94% or even 95% can be achieved as compared to prior art processes that typically achieve a starch conversion of up to 90%. It is believed, without being bound to any particular theory, that LOF starch conversion and yield are higher than for prior art processes because germ has been removed from LOF. Germ contains fermentable starch, but at least a portion of it is bound with protein thereby reducing bioavailability.

As described above, in a preferred embodiment the fermentation medium comprises LOF and added FAN.

In another preferred embodiment the fermentation medium comprises LOF, added FAN and recycled backset (as described above).

In another preferred embodiment, the fermentation medium comprises LOF, added FAN, optionally added backset, and the fermentation is aerated (as described above).

In yet another preferred embodiment, the fermentation medium comprises LOF, added FAN, optionally added backset, the fermentation is optionally aerated, and the yeast addition is split (as described above).

In another preferred embodiment, the fermentation medium comprises LOF, added FAN, optionally added backset, the fermentation is optionally aerated, the yeast addition is optionally split, and the fermentation medium further comprises SEHOF (as described above),

In another preferred embodiment, the fermentation medium comprises LOF, added FAN, optionally added backset, the fermentation is optionally aerated, the yeast addition is optionally split, the fermentation medium optionally further comprises SEHOF, and the FAN addition is metered at a controlled rate (as described above).

In yet another preferred embodiment, the fermentation medium comprises LOF, added FAN, optionally added backset, the fermentation is optionally aerated, the yeast addition is optionally split, the fermentation medium optionally further comprises SEHOF, the FAN addition is optionally metered at a controlled rate, and the glucoamylase addition is metered at a controlled rate (as described above).

In any of the preferred embodiments, a yeast strain capable of metabolism at high ethanol concentration, for example 18 volume percent, 19 volume percent or even 20 volume percent is preferred, and yeast food is preferably added to the fermentation medium.

### Ethanol and DDGS Isolation

After the fermentation is complete, the fermentation composition is fed to a beer well for temporary storage. The fermentation composition is then fed to a reboiler where ethanol and volatile impurities such as fusel oil are separated by vaporization in a distillation column leaving liquid reboiler bottoms (87) (containing dissolved solids).

Fusel oil is a mixture comprising mainly amyl alcohols and lesser amounts of ethyl, propyl, butyl, hexyl and heptyl alcohols, acetaldehydes, and ethyl acetate. It is believed that fusel oil is generated when non-starch components, predominantly present in the germ fraction, such as hemicellulose and pectic are hydrolyzed, and when amino acids undergo reductive deamination. In typical prior art ethanol fermentation processes, about 0.5 to about 1 g/L of fusel oil is generated whereas the process of the present invention, using LOF as feedstock, results in a 30%, 40%, 50%, 60%, 70%, 80% or even 90% reduction in fusel oil content in the ethanol as compared to prior art processes. Fusel oil is predominantly separated and removed from ethanol by distillation. In distillation, the fusel oil components form azeotropes with water, the azeotropes boiling at lower temperatures than water but at higher temperatures than ethanol. Fusel oil is removed from the distillation column in a side-draw located below the column feed inlet. Typically, distillation columns are operated with high dilution to enable efficient removal of fusel oils. Disadvantageously, operation at high dilution is energy intensive and reduces throughput through the distillation operation. Low fusel oil content enables distillation column operation at low dilution resulting in energy savings and throughput increase. It is believed that the net energy input required to produce a liter of ethanol is reduced from 1% to 10% as compared to a reference fermentation process, the reference fermentation process being devoid of LOF and instead comprising whole corn, but otherwise identical to the LOF fermentation process.

The ethanol is condensed and purified in the distillation column. The liquid ethanol exits the top of the distillation column at about 95% purity from where it passes through a molecular sieve dehydration column which removes at least about 75%, 80%, 85%, 90%, 95% or even 99% of the remaining residual water. Fusel oil is hygroscopic and carries about 2 grams of water forward in the ethanol for each gram of fusel oil. The process of the present invention using LOF as feedstock results in lowered fusel oil concentration and decreased water in the alcohol sent to the molecular sieves. It is believed that if fusel oil concentration is reduced by about 50%, the water load on the molecular sieves may decrease by about 3%.

The liquid reboiler bottoms are fed to a centrifuge in order to separate the insoluble solids (DGS) from the liquid centrifugate. The centrifugate is fed to one or more evaporators in an evaporation step in order to boil away moisture leaving a thick syrup which, in prior art processes, typically contains from about 30 to about 40 percent by weight soluble (dissolved) solids (“DS”) from the fermentation. In one embodiment, that concentrated syrup can be mixed with the wet DGS. The wet mixture is termed Distillers Wet Grain with Solubles (DWGS). In another embodiment, the wet DGS is dried to generate DDG. DWGS and DDG are typically used as dairy and beef cattle feed.

It has been discovered that the low non-fermentable content and low solids content of LOF, by virtue of germ and oil removal in fractionation, results in improvements in centrifuge operation as compared to prior art processes. It is believed that the presence of germ and oil interferes with centrifuge separation efficiency because germ has a small particle size and a specific gravity close to the liquid phase specific gravity, and oil can function as an emulsifier and surfactant thereby inhibiting solid-liquid separation. Based on experimental evidence to date, it has been found that centrifuge torque decreases by about 10% when LOF still bottoms are processed as compared to prior art processes. Further, total LOF solids are reduced by about 15% to about 25% as compared to prior art processes. As a direct result of the increase in centrifuge efficiency afforded by LOF as compared to prior art processes, throughput through a centrifuge can be increased by about 25%, or, alternatively, some of the centrifuges in a process can be shut down.

In one embodiment, the centrifugate (117) can be recycled back to the mix tank (20), yeast propagator (50) and/or fermenter (60) as backset as described above.

In another embodiment, the DGS and concentrated syrup mixture may be dried in a drying step to generate DDGS, which is also typically used as dairy and beef cattle feed. The present invention yields DDGS prepared from LOF containing, on an anhydrous basis: about 7 to about 9 percent by weight total oil; from about 35 to about 60 percent by weight total protein, for example, about 35, 40, 45, 50, 55 or even 60 percent by weight total protein; from about 7 to about 11 percent by weight acid detergent fiber (“ADF”), for example, about 7, 8, 9, 10 or even 11 weight percent ADF; from about 15 to about 35 percent by weight neutral detergent fiber (“NDF”), for example, about 15, 20, 25, 30 or even 35 weight percent NDF; from about 1 to about 4 percent by weight ash, for example, 1, 2, 3 or even 4 weight percent ash; and from about 3 to about 10 percent by weight starch, for example, 3, 4, 5, 6, 7, 8, 9 or even 10 percent by weight starch. Experimental evidence to date indicates that the DDGS of the present invention has an angle of repose of about 48° as compared to a value of about 55° for prior art DDGS. That advantage improves flowability and reduces bridging of DDGS in silos, bins and the like.

As compared to prior art processes (based on an LOF fermentation having an equivalent final titer as a whole grain process), it is believed that the process of the present invention would result in a low yield of DDGS as measured in grams of DDGS produced per gram of feed material, primarily as a result of the reduced non-fermentable content in LOF. DDGS yield, based on material balance calculations for grams of DDGS per grams of LOF, would be typically from about 0.15 to about 0.25, for example, about 0.15, 0.2 or 0.25. In another yield measure, as compared to yellow number 2 corn, DDGS yield from LOF would be decreased by about 20%, about 25%, about 30%, about 35%, about 40%, about 45% or even about 50%.

It is believed that the compositional characteristics of the LOF used in the process of the present invention would result in changes to the reboiler bottoms (87), centrifugate (117), syrup (135), wet DGS (115) and DDGS (125) flow rates and composition that alter flow rates to and operational efficiency of the centrifuge (110), dryer (120) and evaporator (130). The net result would be a decrease in overall fermentation process energy usage and emissions. In particular, LOF generates a fermentation composition (65) having a low solids content as compared to prior art processes resulting in an increased ratio of centrifugate to DGS and a concomitant reduced percentage of the process liquid passing to the dryer as a component of wet DGS, and an increased percentage of the process liquid passing to the evaporator as centrifugate. An estimate of percent changes in process flows for ethanol prepared from LOF as compared to ethanol prepared from whole corn (based on a constant ethanol titer), indicates that centrifugate (117) flow to the evaporator (on a unit volume of reboiler bottoms (87) basis) would increase by a factor of up to about 10% by volume for LOF fermentation. Centrifugate flow would increase primarily because reboiler bottoms prepared from LOF would contain a lower solids (i.e., wet DGS) loading as compared to prior art processes. Further, the reduced solids loading would result in a reduction in wet DGS (115) flow of from about 10% to about 50%. Still further, as compared to prior art processes, the LOF of the present invention would generate a fermentation composition (65) characterized by low oil, low glycerol and low fouling salt content. That composition would enable efficient evaporator operation resulting in syrup (135) prepared from LOF having up to 50%, 60% or even 70% DS as compared to up to about 40% to 50% DS in prior art processes. As a result, syrup flow to the dryer (120) would be reduced from about 20% to about 80%, for example 20%, 30%, 40%, 50%, 60%, 70% or even 80%. The net result would be a greater amount of water removed in the evaporator thereby reducing moisture removal requirements on the dryer. It is well known in the art that evaporator efficiency is greater than dryer efficiency, by an order of about 100% to about 300%. Experimental evidence to date indicates that dryer steam usage decreases by about 8% per kilogram of DDG generated from LOF as compared to a kilogram of DDG generated from standard corn to achieve a baseline DDG moisture content of 64%. Based on the increased evaporator loading and decreased dryer loading as compared to prior art processes, experimental evidence to date indicates that the present invention provides total steam usage reductions as compared to steam usage in prior art processes on the order of about 1% to about 20%, for example 1%, 5%, 10%, 15% or even 20%.

It is further believed that the process of the present invention would result in a 5%, 10%, 15%, 20%, 25%, 30%, 35%, 40%, 45% or even 50% reduction in the amount of volatile organic compounds (“VOCs”) emitted on a unit weight or unit volume of ethanol basis as compared to prior art processes. VOCs generated in the fermentation process include ethanol, glycerol, acetic acid, lactic acid, propionic acid, succinic acid and fusel oil. The VOCs are present as components of the DGS and syrup aqueous phase and about 75% of the VOCs are emitted from the fermentation process in the drying step. Prior art fermentation processes typically emit about 3.5 grams of VOCs in the drying step per kilogram of ethanol produced. Because the DGS flow to the dryer in the process of the present invention is reduced by about 10% to about 50%, it is believed that dryer VOC emissions could be reduced to about 3.2, 3, 2.5, 2 or even 1.7 grams of VOCs per kilogram of ethanol produced. In addition to VOCs, drying produces carbon monoxide (CO), which prior art processes typically generate at a rate of about 2.3 grams per kilogram of ethanol produced. Based on reduction in the DGS flow to the dryer, it is believed that the process of the present invention could reduce CO emissions to about 2.1, 2, 1.5 or even 1.1 grams of CO per kilogram of ethanol produced.

In many cases, air permitting regulations mandate reducing VOC emissions below that generated by the fermentation process. Emission reductions require capital investment in pollution control equipment such as, for example, scrubbers, condensers, carbon beds and/or thermal oxidizers. The VOC reductions provided by the present process enable capital investment to be avoided in some cases, and in other cases the pollution control equipment can be operated with increased efficiency.

Advantageously, the reduced DGS flow afforded by the present process reduces or avoids capital investment expenditures for solid-liquid separation (e.g., centrifuge) and drying equipment by virtue of reduced material processing capacity needs. Importantly, in existing fermentation facilities that are constrained by drying capacity but not by fermentation capacity, the process of the present invention can effectively increase throughput without capital investment in solid-liquid separation (e.g., centrifuge), drying and pollution control equipment.

Having described the invention in detail, it will be apparent that modifications and variations are possible without departing from the scope of the invention defined in the appended claims.

### EXAMPLES

The following non-limiting examples are provided to further illustrate the present invention.

### Example 1

In laboratory dry grind ethanol fermentations with a low oil corn fraction (LOF) prepared by fractionating whole corn in a Buhler-L fractionator at a ratio of LOF to high oil fraction (HOF) of about 63:37, the effect of added urea levels of 1.2 mg urea/g starch (1×), 1.5 mg urea/g starch (1.25×), 1.8 mg urea/g starch (1.5×), 2.1 mg urea/g starch (1.75×), 2.4 mg urea/g starch (2×) and 3.6 mg urea/g starch (3×) on fermentation rate were evaluated according to the following procedure. A LOF mash having 31.1% w/w solids (28% dry basis (“DB”)) was prepared by grinding 1213.0 grams of LOF through a 0.75 mm screen and then adding it to 2688 g deionized (“DI”) water with agitation in a tared beaker. 590 μL Spezyme Fred α-amylase and 0.62 g of CaCl₂ were added. The temperature was raised to 90° C., held at that temperature for 25 minutes, and then cooled to 40° C. 1.0 mL of glucoamylase (Distillase L-400) was then added. An Ethanol Red (Fermentis) yeast suspension was prepared by combining 4.2 g yeast and 16.7 g sterile water, and gently shaking for about 15 minutes. 14.4 mL of the yeast was added to the LOF mash. The mash contained 26.1% dry solids and was 12.4 DE. 3.8 g Wyeast nutrient was added to 60 ml of water and boiled for about 10 minutes. 56 ml of the prepared nutrient was added to the LOF mash followed by 1.8 mL of a 10 mg/mL V50 antibiotic solution. 300 g were dispensed to each of 12 flasks. Urea solution was added to each flask resulting in duplicates at each urea concentration of 1.2 mg urea/g starch (1×), 1.5 mg urea/g starch (1.25×), 1.8 mg urea/g starch (1.5×), 2.1 mg urea/g starch (1.75×), 2.4 mg urea/g starch (2×) and 3.6 mg urea/g starch (3×). The flasks were placed on a rotary shaker at 125 rpm and were maintained at 32° C. Each flask was sampled twice per day and analyzed for pH, ethanol (by HPLC), carbohydrate (by HPLC), yeast count and viability. For each urea concentration, ethanol yield (in grams ethanol per liter) at 18, 26, 42, 50 and 56.5 hours is reported in Table 1A. Ethanol productivity (in grams of ethanol per liter per hour) is reported in Table 1B and carbohydrate utilization at those times is reported in Table 1C. Glycerol concentration in g/L is reported in Table 1D and DP4 concentration in g/L is reported in Table 1E.

TABLE A
Ethanol Yield (g ethanol/L)

| Urea | 18 Hours | 26 Hours | 42 Hours | 50 Hours | 56.5 Hours |
|---|---|---|---|---|---|
| 1X | 48.7 | 62.5 | 76.2 | 89.0 | 99.8 |
| 1.25X | 51.9 | 66.5 | 85.5 | 94.4 | 106.4 |
| 1.5X | 56.5 | 70.1 | 96.0 | 98.4 | 108.1 |
| 1.75X | 56.8 | 72.8 | 97.3 | 102.4 | 111.4 |
| 2X | 59.0 | 76.6 | 102.0 | 105.6 | 111.2 |
| 3X | 66.4 | 87.6 | 111.5 | 109.1 | 114.1 |

TABLE 1B
Ethanol Productivity (g ethanol/L/hr)

| Urea | 18 Hours | 26 Hours | 42 Hours | 50 Hours | 56.5 Hours |
|---|---|---|---|---|---|
| 1X | 2.71 | 2.40 | 1.81 | 1.78 | 1.77 |
| 1.25X | 2.88 | 2.56 | 2.04 | 1.89 | 1.88 |
| 1.5X | 3.14 | 2.70 | 2.29 | 1.97 | 1.91 |
| 1.75X | 3.16 | 2.80 | 2.32 | 2.05 | 1.97 |
| 2X | 3.28 | 2.95 | 2.43 | 2.11 | 1.97 |
| 3X | 3.69 | 3.37 | 2.65 | 2.18 | 2.02 |

TABLE 1C
Carbohydrate Utilization^{a}

| Urea | 18 Hours | 26 Hours | 42 Hours | 50 Hours | 56.5 Hours |
|---|---|---|---|---|---|
| 1X | 93.5 | 80.2 | 25.1 | 13.2 | 2.8 |
| 1.25X | 92.1 | 71.5 | 18.0 | 5.3 | 0 |
| 1.5X | 85.2 | 63.0 | 7.9 | 3.7 | 0 |
| 1.75X | 76.8 | 55.8 | 7.2 | 3.6 | 0 |
| 2X | 73.5 | 52.7 | 6.1 | 3.1 | 0 |
| 3X | 69.9 | 55.3 | 3.0 | 1.6 | 0 |

^{a}Carbohydrate utilization is reported as the carbohydrate concentration remaining in solution in g/L.

TABLE 1D
Glycerol Concentration (g/L)

| Urea | 18 Hours | 26 Hours | 42 Hours | 50 Hours | 56.5 Hours |
|---|---|---|---|---|---|
| 1X | 5.2 | 6.3 | 6.3 | 7.8 | 7.8 |
| 1.25X | 5.4 | 6.5 | 6.8 | 7.8 | 7.6 |
| 1.5X | 5.7 | 6.6 | 8.2 | 8.5 | 8.2 |
| 1.75X | 5.6 | 6.7 | 8.1 | 8.4 | 8.2 |
| 2X | 5.7 | 5.5 | 7.5 | 8.9 | 7.4 |
| 3X | 6.1 | 7.8 | 9.1 | 9.6 | 9.2 |

TABLE 1E
DP4 Concentration (g/L)

| Urea | 18 Hours | 26 Hours | 42 Hours | 50 Hours | 56.5 Hours |
|---|---|---|---|---|---|
| 1X | 71.7 | 59.5 | 13.3 | 7.1 | 2.8 |
| 1.25X | 71.1 | 56.6 | 10.1 | 5.2 | <1 |
| 1.5X | 70.9 | 53.6 | 8 | 3.7 | <1 |
| 1.75X | 68.1 | 52.9 | 7.2 | 3.6 | <1 |
| 2X | 68.5 | 52.8 | 6.1 | 3.1 | <1 |
| 3X | 69.9 | 55.3 | 3 | 1.6 | <1 |

The data show a direct correlation between urea concentration and fermentation rate. At 3× urea, the fermentation finished in 42 hours, whereas 57 hours was required for fermentation for 2× and 1.75× urea. The speed of fermentation can be observed even at 18 hours where the 3× urea fermentation generated 25% more ethanol than did the 1× fermentation.

### Example 2

A series of five fermentations of about 16,000 liters each were run in order to evaluate the fermentation performance of LOF (prepared as described in Example 1) versus degermed yellow number 2 corn (i.e., flaked corn grits). Each fermentation was done in a 23,000 liter fermenter filled to about 16,000 liters and containing about 5450 kg fractionated corn as the carbohydrate source. The fermentations were filled over a 24-hour interval with the yeast pitched into the first incoming mash. The composition of flaked grits and LOF is described in Table 2A below. The operating parameters for the fermentations is described in Table 2B below. Carbohydrate utilization (i.e., consumption) is described in Table 2C, below, and ethanol yield (grams of ethanol per liter) is described in Table 2D, below. Ethanol productivity (grams ethanol per liter per hour) is described in Table 2E, below. Final productivity and ethanol yield from starch for the five fermentations were compared with laboratory fermentation results for LOF and yellow number 2 corn, with the results described in Table 2F, below. Final productivity, or rate, was calculated by dividing the final ethanol titer by the fermentation residence time. Fermentations 1, 2, 4 and 5 reached a final (maximum) ethanol titer in 55 hours, while fermentation 3 reached the final ethanol concentration at 50 hours. The final, 55 hour, fermenter beer HPLC results are reported in Table 2G.

TABLE 2A

| Component (% w/w) | Flaked Grits | LOF |
|---|---|---|
| Moisture | 11.5 | 9.5 |
| Starch | 71.9 | 69.8 |
| Starch (DB) | 81.2 | 77.1 |
| Crude Fat (oil) | 0.6 | 1.8 |
| Crude Fat (DB) | 0.7 | 2.0 |
| Crude Protein | 7.3 | 7.8 |
| Crude Protein (DB) | 8.2 | 8.6 |

TABLE 2B

| Parameter | Ferm. 1 | Ferm. 2 | Ferm. 3 | Ferm. 4 | Ferm. 5^{d} |
|---|---|---|---|---|---|
| Corn Source | Flaked | Flaked | LOF | LOF | LOF |
| | Grits | Grits | | | |
| Corn Weight | 5594 | 5446 | 5175 | 5365 | 5049 |
| (kg) | | | | | |
| Suspension | 32.8 | 31.9 | 33.1 | 33.9 | 33.6 |
| TDS % | | | | | |
| Suspension | 5.8 | 5.8 | 5.8 | 5.8 | 5.8 |
| pH | | | | | |
| Suspension | 8.4 | 7.8 | 7.0 | 7.4 | 7.9 |
| DE | | | | | |
| Mash pH | 5.9 | 6.0 | 5.9 | 5.9 | 5.9 |
| Mash DE | 17.3 | 14.0 | 10.4 | 10.2 | 10.4 |
| Wort TDS % | 29.9 | 30.0 | 31.8 | 32.1 | 32.4 |
| Wort pH | 5.3 | 6.1 | 5.9 | 5.8 | 5.9 |
| Wort DS | no data | 21.4 | 21.7 | 21.8 | 23.3 |
| α-amylase | 140 | 140 | 140 | 140 | 140 |
| TA-0990 | | | | | |
| (g/hr)^{a} | | | | | |
| α-amylase | 65 | 0 | 0 | 0 | 0 |
| TA-0950 | | | | | |
| (g/hr) | | | | | |
| Gluco- | 6990 | 6990 | 6820 | 5740 | 5660 |
| amylase | | | | | |
| dose (g) | | | | | |
| Urea | 1.12 mg/g | 1.12 mg/g | 1.12 mg/g | 1.12 mg/g | 1.12 mg/g |
| | starch | starch | starch | starch | starch |
| Volume (L) | 15,974 | 15,895 | 15,899 | 15,899 | 15,903 |
| Weight (kg) | 17,345 | 17,397 | 17,588 | 17,544 | 17,510 |
| Aeration | 220 | 220 | 220 | no air | no air |
| Rate (SLM)^{b} | | | | | |
| Aeration | 7.8 | 5.0 | 9.9 | — | — |
| time (hours) | | | | | |
| L 15% | 28.5 | 34.0 | 11.3 | 3.8 | — |
| NH₄OH^{c} | | | | | |

^{a}0.6 g α-amylase per kg LOF was added
^{b}Standard liters per minute
^{c}Liters of 15% ammonium hydroxide added
^{d}The yeast pitch to Fermentation 5 was split with one half added at 0 time and the remainder at 6 hours.

TABLE 2C
Carbohydrate Utilization^{a}

| Hours | Ferm. 1 | Ferm. 2 | Ferm. 3 | Ferm. 4 | Ferm. 5 |
|---|---|---|---|---|---|
| 3 | 240 | 240 | 247 | 250 | 250 |
| 7 | 228 | 228 | 200 | 211 | 243 |
| 14 | 182 | 184 | 170 | 151 | 150 |
| 20 | 152 | 137 | 122 | 127 | 143 |
| 26 | 122 | 111 | 103 | 107 | 106 |
| 31 | 83 | 81 | 69 | 78 | 74 |
| 37 | 61 | 54 | 38 | 52 | 55 |
| 44 | 34 | 30 | 14 | 34 | 36 |
| 50 | 18 | 11 | 2 | 18 | 16 |
| 56^{b} | 7 | 0 | 0 | 5 | 7 |

^{a}Carbohydrate utilization is reported as the carbohydrate concentration remaining in solution in g/L.
^{b}The final measurement for fermentations 3-5 was at 56 hours.

TABLE 2D
Ethanol Yield (g ethanol/L)

| Hours | Ferm. 1 | Ferm. 2 | Ferm. 3 | Ferm. 4 | Ferm. 5 |
|---|---|---|---|---|---|
| 3 | 7 | 5 | 7 | 5 | 5 |
| 7 | 26 | 29 | 31 | 28 | 21 |
| 14 | 42 | 42 | 50 | 51 | 47 |
| 20 | 56 | 58 | 63 | 62 | 60 |
| 26 | 69 | 69 | 74 | 74 | 73 |
| 31 | 86 | 86 | 87 | 90 | 88 |
| 37 | 97 | 100 | 105 | 102 | 99 |
| 44 | 110 | 109 | 114 | 109 | 108 |
| 50 | 115 | 117 | 122 | 117 | 117 |
| 54^{a} | 121 | 120 | 123 | 124 | 122 |

^{a}The final measurement for fermentations 3-5 was at 56 hours.
^{b}Fermentation 3 finished in 50 hours when a final ethanol concentration of 124 g/L was reached, while fermentations 4 and 5 finished reached a final concentration of 124 g/L at 55 hours.

TABLE 2E
Ethanol Productivity (g ethanol/L/hr)

| Hours | Ferm. 1 | Ferm. 2 | Ferm. 3 | Ferm. 4 | Ferm. 5 |
|---|---|---|---|---|---|
| 3 | 2.33 | 1.67 | 2.33 | 1.67 | 1.67 |
| 7 | 3.71 | 4.14 | 4.43 | 4 | 3 |
| 14 | 3 | 3 | 3.57 | 3.64 | 3.36 |
| 20 | 2.8 | 2.9 | 3.15 | 3.1 | 3 |
| 26 | 2.65 | 2.65 | 2.85 | 2.85 | 2.81 |
| 31 | 2.77 | 2.77 | 2.81 | 2.9 | 2.84 |
| 37 | 2.62 | 2.7 | 2.84 | 2.76 | 2.68 |
| 44 | 2.5 | 2.48 | 2.59 | 2.48 | 2.45 |
| 50 | 2.3 | 2.34 | 2.44 | 2.34 | 2.34 |
| 54^{a} | 2.24 | 2.22 | 2.19 | 2.21 | 2.18 |

^{a}The final measurements for fermentations 3-5 was 56 hours.

TABLE 2F
Ethanol Productivity and Ethanol Yield on a Starch
Basis

| | Ethanol Production | Ethanol Yield |
|---|---|---|
| | (g ethanol/L/hr) | (g/g starch) |
| Flaked Grits (Ferm. 1) | 2.20 | 0.48 |
| Flaked Grits (Ferm. 2) | 2.18 | 0.49 |
| LOF (Ferm. 3) | 2.46 | 0.53 |
| LOF (Ferm. 4) | 2.25 | 0.52 |
| LOF (Ferm. 5) | 2.22 | 0.56 |
| Lab Yellow #2 Corn | 2.13 | 0.54 |
| Lab LOF | 2.29 | 0.47 |

TABLE 2G
Final HPLC Fermenter Beer Results

| Analyte | | | | | |
|---|---|---|---|---|---|
| (g/L) | Ferm. 1 | Ferm. 2 | Ferm. 3 | Ferm. 4 | Ferm. 5^{c} |
| Corn Source | Flaked | Flaked | LOF | LOF | LOF |
| | Grits | Grits | | | |
| DP4 | 2 | <2 | <2 | 2 | 3 |
| DP3 | <2 | <2 | <2 | <2 | <2 |
| DP2 | 2 | <2 | <2 | <2 | <2 |
| Glucose | 3 | <2 | <2 | 3 | 4 |
| Total CHO | 7 | <2 | <2 | 5 | 7 |
| Lactic Acid | 1.4 | 1.6 | 1.3 | 1.2 | 1.4 |
| Acetic Acid | 1.3 | 1.2 | 0.4 | 0.2 | 0.3 |
| Glycerol | 15 | 13 | 12 | 11 | 11 |
| Ethanol | 121 | 120 | 123 | 124 | 122 |

Ethanol fermentations with both flaked grits and LOF were completed in 55 hours, with the exception of LOF fermentation 3 that finished in about 50 hours. Final ethanol concentration was about 120 g/L and ethanol yield (g ethanol/g starch) ranged from about 0.48 to about 0.56.

A pH drop in the fermenting mash during the first 10 hours of run time reached lower levels than in the laboratory experiments. Ammonium hydroxide was added to adjust the pH. The pH generally rose to about 3.5 after about 20 hours and remained at that point for the remainder of the fermentation. The pH drop was less severe for LOF than for flaked grits and was further suppressed by eliminating aeration and splitting the yeast pitch with about half of the yeast added at time 0 and the remainder added at 6 hours of fermentation time. Elimination of aeration and splitting the yeast pitch were successful in maintaining adequate pH, but the residence time was extended by about five hours as a result.

Fermenter carbohydrate consumption was somewhat different in the 16,000 L fermentations than in laboratory experiments. The difference can be attributed to the fermenter filling method. The 16,000 L vessels were filled over a 24 hour interval, with the yeast pitched into the first incoming mash. In contrast, the laboratory experiments were performed by pitching the yeast into full fermenters. A consequence of the 16,000 L method is that there was continuous dilution with mash containing hydrolysate. Dextrose levels fell to less than 1.0 g/L by 14-20 hours as the yeast culture outpaced the activity of the glucoamylase enzyme. A supplemental dose of Distillate-400, amounting to about 50% in excess of theoretical was added in order to allow adequate carbohydrate for unimpaired fermentation.

Fermentation 3 finished 5 hours ahead of the other fermentations. As compared to LOF fermentations 4 and 5, that fermentation was aerated for 10 hours, received the highest total amount of glucoamylase and did not have a split yeast pitch.

### Example 3

Four commercial yeast strains were evaluated for use in fermenting LOF (prepared as described in Example 1) mash, wherein a mash containing 29 percent by weight LOF, having a DE value of 13.9, but wherein each mash contained 3.6 mg urea/g starch (3×), was prepared. Yeast strains evaluated included ETHANOL RED (available from Red Star/Lesaffre, USA); BioFerm HP and XR (available from North American Bioproducts); and SUPERSTART (available from Lallemand). One experiment further evaluated ETHANOL RED in the above described mash, but containing 6 mg urea/g starch (5×).

The yeasts were prepared in sterile water at 1.25 grams yeast per 5 mL water followed by a 15 minute incubation with shaking. The yeasts were diluted and counted with the results reported in Table 3A.

TABLE 3A

| Yeast | Cells/Dry Gram | Cells/mL mash at Time 0 |
|---|---|---|
| Ethanol Red | 3.4 × 10¹⁰ | 37 × 10⁶ |
| Superstart | 1.8 × 10¹⁰ | 15 × 10⁶ |
| Bioferm HP | 3.7 × 10¹⁰ | 37 × 10⁶ |
| Bioferm XR | 3.4 × 10¹⁰ | 37 × 10⁶ |

Ethanol titer for the yeasts, in grams ethanol per liter, is reported in Table 3B where fermentation 1 represents ETHANOL RED (3× urea), fermentation 2 represents ETHANOL RED (5× urea), fermentation 3 represents SUPERSTART, fermentation 4 represents Bioferm HP and fermentation 5 represents Bioferm XR. Carbohydrate utilization is shown in Table 3C and fermentation pH is shown in Table 3D.

TABLE 3B
Ethanol Titer (g ethanol/L)

| Ferm | 18 Hours | 25 Hours | 41 Hours | 49 Hours | 65 Hours |
|---|---|---|---|---|---|
| 1 | 73.0 | 88.4 | 107.5 | 123.9 | 128.4 |
| 2 | 81.8 | 98.8 | 119.2 | 130.1 | 130.4 |
| 3 | 70.6 | 80.9 | 93.1 | 106.7 | 107.2 |
| 4 | 74.4 | 94.6 | 120.0 | 123.9 | 126.1 |
| 5 | 71.3 | 90.9 | 114.7 | 123.9 | 128.4 |

TABLE 3C
Carbohydrate Utilization^{a}

| Ferm | 18 Hours | 25 Hours | 41 Hours | 49 Hours | 65 Hours |
|---|---|---|---|---|---|
| 1 | 84.0 | 68.1 | 22.6 | 0 | 0 |
| 2 | 83.4 | 63.8 | 2.1 | 0 | 0 |
| 3 | 79.3 | 54.4 | 22.7 | 0 | 0 |
| 4 | 83.3 | 67.6 | 6.5 | 0 | 0 |
| 5 | 80.9 | 66.1 | 6.9 | 0 | 0 |

^{a}Carbohydrate utilization is reported as the carbohydrate concentration remaining in solution in g/L.

TABLE 3D
Fermentation pH

| Ferm | 18 Hours | 25 Hours | 41 Hours | 49 Hours | 65 Hours |
|---|---|---|---|---|---|
| 1 | 3.28 | 3.25 | 3.45 | 3.64 | 3.75 |
| 2 | 3.36 | 3.35 | 3.72 | 3.94 | 4.12 |
| 3 | 3.35 | 3.29 | 3.58 | 3.78 | 3.90 |
| 4 | 3.27 | 3.21 | 3.53 | 3.68 | 3.77 |
| 5 | 3.33 | 3.28 | 3.51 | 3.69 | 3.79 |

ETHANOL RED, Bioferm HP and Bioferm XR performed similarly in LOF fermentations and the highest ethanol achieved was 124 grams ethanol per liter in 49 hours. SUPERSTART achieved 106 grams ethanol per liter. The number of viable cells per gram was lower for SUPERSTART which may have affected the fermentation results. The pH profiles of the yeast were similar with the lowest pH measured at 3.2 at 25 hours.

### Example 4

Ethanol fermentations were done with corn and LOF mashes as low (27 percent by weight solids) and high solids (34 percent by weight solids) loading in order to evaluate whether high solids fermentation could be used.

An LOF mash having 27% w/w solids was prepared by grinding 305.0 grams of LOF (prepared as described in Example 1) from Example 1 through a 0.75 mm screen and then adding it to 712 g deionized (“DI”) water with agitation in a tared beaker. 153 μL Spezyme Fred and 0.16 g of CaCl₂ were added. The temperature was raised to 90° C., held at that temperature for 25 minutes, and then cooled to 40° C. The pH was adjusted to 5 to 5.2 with sulfuric acid. 262 μL of glucoamylase (Distillase L-400) was then added. An Ethanol Red (Fermentis) yeast suspension was prepared by combining 9.0 g yeast and 33.4 g sterile water, and gently shaking for about 15 minutes. 6.5 mL of the yeast suspension was added to the LOF mash. Urea was added at a ratio of about 1.1 mg urea per gram of starch. 3.8 grams Wyeast nutrient and 0.4 mL of a 10 mg/mL V50 antibiotic solution was added to the mash. 300 g were dispensed to each of 3 sterilized flasks. The flasks were placed on a rotary shaker at 125 rpm and were maintained at 32° C. Each flask was sampled twice per day and analyzed for pH, ethanol (by HPLC), carbohydrate (by HPLC), yeast count and viability. A high solids loading LOF mash having 34 percent by weight solids and corn mashes having 27 and 34 percent by weight solids were prepared by similar procedures and proportional mash component concentrations based on starch content. Mash characteristics are reported in Table 4A. Ethanol titer is reported in Table 4B, ethanol productivity is reported in table 4C, carbohydrate utilization is reported in Table 4D and fermentation pH is reported in Table 4E. Impurity concentrations, in g/L, for glycerol, lactic acid, acetic acid, DP2 maltose and DP3 maltotriose at 38, 45 and 61 hour fermentation times are reported in Table 4F. In each table, fermentation 1 is 27% solids LOF, fermentation 2 is 34% solids LOF, fermentation 3 is 28.5% solids corn and fermentation 4 is 32.5% solids corn.

TABLE 4A
Mash Characteristics

| | Ferm. 1 | Ferm. 2 | Ferm. 3 | Ferm. 4 |
|---|---|---|---|---|
| pH | 5.42 | 5.64 | 5.87 | 5.93 |
| DE | no data | 12 | 18 | 14.6 |
| Starch (g/L) | 209.3 | 249.8 | 186 | 221.9 |
| Solids (%) | 27 | 34 | 28.5 | 32.5 |
| Added Urea (mg) | 225 | 292 | 225 | 292 |
| mg urea/g starch | 1.08 | 1.17 | 1.2 | 1.32 |

TABLE 4B
Ethanol Titer (g ethanol per liter)

| Hours | Ferm. 1 | Ferm. 2 | Ferm. 3 | Ferm. 4 |
|---|---|---|---|---|
| 15 | 50.3 | 57.3 | 71.7 | 77.6 |
| 22 | 65.1 | 78.3 | 83 | 89.8 |
| 38 | 94.5 | 113.5 | 109.3 | 122.7 |
| 45 | 106.1 | 115.7 | 119.8 | 125.2 |
| 61 | 121.8 | 128.3 | 141 | 137.9 |
| 67 | 127.7 | 126 | 133 | 132.6 |
| 83 | 130.7 | 131.2 | 132.4 | 145.1 |

TABLE 4C
Ethanol Productivity (g ethanol per liter per hour)

| Hours | Ferm. 1 | Ferm. 2 | Ferm. 3 | Ferm. 4 |
|---|---|---|---|---|
| 15 | 3.35 | 3.82 | 4.78 | 5.17 |
| 22 | 2.96 | 3.56 | 3.77 | 4.08 |
| 38 | 2.49 | 2.99 | 2.88 | 3.23 |
| 45 | 2.36 | 2.57 | 2.66 | 2.78 |
| 61 | 2 | 2.1 | 2.31 | 2.26 |
| 67 | 1.91 | 1.88 | 1.99 | 1.98 |
| 83 | 1.57 | 1.58 | 1.60 | 1.75 |

TABLE 4D
Carbohydrate Utilization^{a}

| Hours | Ferm. 1 | Ferm. 2 | Ferm. 3 | Ferm. 4 |
|---|---|---|---|---|
| 15 | 68.9 | 73.4 | 44 | 75.8 |
| 22 | 66.7 | 93.2 | 44.2 | 78.5 |
| 38 | 53.3 | 91.1 | 23.1 | 57.9 |
| 45 | 43.3 | 86.3 | 22.9 | 57.9 |
| 61 | 19.9 | 92.8 | 9.8 | 60.8 |
| 67 | 3.3 | 85.9 | 5.4 | 55.2 |
| 83 | 8.9 | 95.9 | 5.5 | 62.1 |

^{a}Carbohydrate utilization is reported as the carbohydrate concentration remaining in solution in g/L.

TABLE 4E
Fermentation pH

| Hours | Ferm. 1 | Ferm. 2 | Ferm. 3 | Ferm. 4 |
|---|---|---|---|---|
| 15 | 3.18 | 3.38 | 3.8 | 4 |
| 22 | 3.03 | 3.25 | 3.64 | 3.87 |
| 38 | 3.06 | 3.45 | 3.75 | 4.12 |
| 45 | 3.18 | 3.59 | 3.82 | 4.18 |
| 61 | 3.33 | 3.72 | 4 | 4.31 |
| 67 | 3.39 | 3.72 | 4.01 | 4.3 |
| 83 | 3.45 | 3.73 | 4.03 | 4.31 |

TABLE 4F
Glycerol, Lactic Acid and Acetic Acid Impurities
and DP2 and DP3 Concentrations (g/L)

| Hours | Impurity | Ferm. 1 | Ferm. 2 | Ferm. 3 | Ferm. 4 |
|---|---|---|---|---|---|
| 38 | glycerol | 6.4 | 9.4 | 7.8 | 9.2 |
| 38 | lactic | 8 | 7.9 | 8.1 | 7.9 |
| 38 | acetic | <1 | <1 | 2.4 | 3.2 |
| 38 | DP2 | <1 | <1 | <1 | <1 |
| 38 | DP3 | <1 | <1 | <1 | <1 |
| 45 | glycerol | 7 | 9 | 11.6 | 10.1 |
| 45 | lactic | 8.3 | 7.9 | 8.4 | 8.1 |
| 45 | acetic | 3.2 | 3 | 3.6 | 3.1 |
| 45 | DP2 | <1 | <1 | <1 | <1 |
| 45 | DP3 | <1 | <1 | <1 | <1 |
| 61 | glycerol | 8.6 | 12.7 | 10 | 11.2 |
| 61 | lactic | 7.9 | 7.9 | 7.7 | 7.9 |
| 61 | acetic | 3 | 3.1 | 3.3 | 3.3 |
| 61 | DP2 | <1 | <1 | <1 | <1 |
| 61 | DP3 | <1 | <1 | <1 | <1 |

Measured ethanol was high in the four fermentations with values of about 130 grams per liter to grams per liter achieved. The fermentations were essentially finished by about 67 hours. The fermentation time was generally slower than 55 hours. It is believed that the relative low ratio of FAN to starch in the LOF fermentations (about 1.1:1 to about 1.2:1 on a gram of urea per gram of starch basis) caused those fermentations to be nitrogen limited and to run slower than the yellow number 2 corn fermentations. Although the yellow number 2 corn fermentations contained a similar added FAN content (about 1.2:1 to about 1.3:1 on a gram of urea per gram of starch basis), the corn fermentations contained a higher level of total FAN than the LOF fermentations because corn mashes contain high germ content as compared to LOF mashes and it is known that germ is a FAN source.

### Example 5

Ethanol fermentations were done with corn and LOF mashes to evaluate the effect of using various levels of backset in fermentations as a yeast food replacement, including replacing 25%, 50% and 100% of the added water, on a volume basis, with backset.

A LOF mash having 27% w/w solids (DB) and 25% backset was prepared by grinding 300.0 grams of LOF (prepared as described in Example 1) (209.4 g starch) through a 0.75 mm screen and then adding it to 175 grams backset (containing 3.4 percent by weight solids) and 525 g DI water with agitation in a tared beaker. 146.6 μL Spezyme Fred L and 0.154 g of CaCl₂ were added. The pH was adjusted to 5.5. The temperature was raised to 90° C., held at that temperature for 25 minutes, and then cooled to 40° C. 251 μL of glucoamylase (Distillase L-400) was then added. An Ethanol Red (Fermentis) yeast suspension was prepared by combining 9.0 g yeast and 33.4 g sterile water, and gently shaking for about 15 minutes. 3.6 mL of the yeast suspension was added to the LOF mash. Urea was added at a ratio of 2.2 mg urea per gram of starch. 0.43 mL of a 10 mg/mL V50 antibiotic solution was added to the mash. 300 g were dispensed to each of 2 sterilized flasks. The flasks were placed on a rotary shaker at 125 rpm and were maintained at 32° C. Each flask was sampled twice per day and analyzed for pH, ethanol (by HPLC), carbohydrate (by HPLC), yeast count and viability. LOF fermentations having 50% and 100% backset, and corn mashes having 25%, 50% and 100% backset were prepared by similar procedures and proportional mash component concentrations based on starch content. Mash characteristics are reported in Table 5A. Ethanol titer is reported in Table 5B, ethanol productivity is reported in table 5C, carbohydrate utilization is reported in Table 5D and fermentation pH is reported in Table 5E. Fermentation Impurity Content (g/L of glycerol, DP2 dextrose, and DP4 dextrose) is reported in Table 5F. In each table, fermentation 1 is a LOF mash containing 25% backset, fermentation 2 is a LOF mash containing 50% backset, fermentation 3 is a LOF mash containing 100% backset, fermentation 4 is a corn mash containing 25% backset, fermentation 5 is a corn mash containing 50% backset and fermentation 6 is a corn mash containing 100% backset.

TABLE 5A
Mash Characteristics

| | Ferm. 1 | Ferm. 2 | Ferm. 3 |
|---|---|---|---|
| pH | 5.4 | 5.4 | 5.3 |
| DE | 14.5 | 16.3 | 20.9 |
| Solids (%) | 24.4 | 23.9 | 24.9 |
| mg urea/g | 2.2 | 2.2 | 2.2 |
| starch | | | |
| | Ferm. 4 | Ferm. 5 | Ferm. 6 |
| pH | 5.6 | 5.4 | 5.4 |
| DE | 15.9 | 18.9 | 23.3 |
| Solids (%) | 25 | 24.9 | 25.2 |
| mg urea/g | 2.2 | 2.2 | 2.2 |
| starch | | | |

TABLE 5B
Ethanol Titer (g ethanol per liter)

| Hours | Ferm. 1 | Ferm. 2 | Ferm. 3 |
|---|---|---|---|
| 17 | 47.4 | 56.8 | 63.5 |
| 24 | 61.5 | 71.6 | 80.9 |
| 41 | 98.8 | 102.7 | 106.7 |
| 48 | 106.1 | 106.7 | 107.5 |
| 67 | 110.9 | 106.6 | 106.2 |
| 92 | 107.9 | 105.1 | 105.6 |
| Hours | Ferm. 4 | Ferm. 5 | Ferm. 6 |
| 17 | 43.8 | 53.9 | 57.6 |
| 24 | 56.8 | 71.5 | 82.8 |
| 41 | 90.8 | 103.3 | 108.2 |
| 48 | 98.5 | 108.1 | 109.9 |
| 67 | 107 | 109.1 | 109.5 |
| 92 | 107.1 | 108.8 | 109.1 |

TABLE 5C
Ethanol Productivity (g ethanol per liter per hour)

| Hours | Ferm. 1 | Ferm. 2 | Ferm. 3 |
|---|---|---|---|
| 17 | 2.79 | 3.34 | 3.74 |
| 24 | 2.56 | 2.98 | 3.37 |
| 41 | 2.41 | 2.5 | 2.6 |
| 48 | 2.21 | 2.22 | 2.24 |
| 67 | 1.66 | 1.59 | 1.59 |
| 92 | 1.17 | 1.14 | 1.15 |
| Hours | Ferm. 4 | Ferm. 5 | Ferm. 6 |
| 17 | 2.58 | 3.17 | 3.39 |
| 24 | 2.37 | 2.98 | 3.45 |
| 41 | 2.21 | 2.52 | 2.64 |
| 48 | 2.05 | 2.25 | 2.29 |
| 67 | 1.6 | 1.63 | 1.63 |
| 92 | 1.16 | 1.18 | 1.19 |

TABLE 5D
Carbohydrate Utilization^{a}

| Hours | Ferm. 1 | Ferm. 2 | Ferm. 3 |
|---|---|---|---|
| 17 | 96.6 | 73.9 | 59.3 |
| 24 | 66.9 | 44.7 | 39.3 |
| 41 | 18.8 | 13.6 | 14.4 |
| Hours | Ferm. 4 | Ferm. 5 | Ferm. 6 |
| 17 | 107.6 | 87.2 | 72.1 |
| 24 | 81 | 66.8 | 68 |
| 41 | 27.6 | 20 | 16.9 |

^{a}Carbohydrate utilization is reported as the carbohydrate concentration (the sum of glucose, DP2, DP3 and DP4) remaining in solution in g/L.

TABLE 5E
Fermentation Impurity Content (g/L of glycerol,
DP2 dextrose, and DP4 dextrose)^{a}

| Hours | Ferm. 1 | Ferm. 2 | Ferm. 3 |
|---|---|---|---|
| 17 | 6.2, 15.7, 64.9 | 8.7, 5.3, 58 | 13.4, 0.83, 57.5 |
| 24 | 7.2, 3.7, 54.2 | 10, <1, 42.7 | 13.5, <1, 37.3 |
| 41 | 8.9, n.d., 17.9 | 10.4, n.d., | 14.6, n.d., 13.4 |
| | | 12.7 | |
| 48 | 8.9, n.d., 12.8 | 10.8, n.d., | 14.6, n.d., 11.7 |
| | | 11.4 | |
| 67 | 9, n.d., 10.4 | 10.5, n.d., | 12, n.d., 10.7 |
| | | 10.4 | |
| 92 | 9.7, n.d., <2.5 | 9.6, n.d., <2.5 | 14.3, n.d., <2.5 |
| Hours | Ferm. 4 | Ferm. 5 | Ferm. 6 |
| 17 | 6.6, 15.7, 66.9 | 8.3, 12.3, 65.3 | 12.2, 10.6, 60.6 |
| 24 | 7.5, 5.1, 57.3 | 9.4, 9.2, 56.5 | 9, 7, 59.9 |
| 41 | 9.2, n.d., 22.5 | 10.5, n.d., 19 | 15, n.d., 15.9 |
| 48 | 9.6, n.d., 16.7 | 10.3, n.d., | 14.6, n.d., 13.6 |
| | | 14.3 | |
| 67 | 9.3, n.d., 11.3 | 10.2, n.d., | 14.5, n.d., 12.9 |
| | | 12.4 | |
| 92 | 8.5, n.d., <2.5 | 10.1, n.d., | 13.9, n.d., 2.7 |
| | | <2.5 | |

^{a}The first reported number is glycerol, the second reported number is DP2 dextrose and the third reported number is DP4 dextrose. n.d. refers to “not detectable.”

All fermentations finished by 48 hours and achieved about 110 g ethanol per liter. The fermentation rates were between about 2.2 and about 2.6 grams ethanol per liter per hour when measured at 41 hours. The addition of backset affected the pH of the mashes and appeared to buffer the pH drop normally seen in LOF fermentations. The pH of the mashes including backset was about 3.6 (for 25% backset) and about 3.8 (for 50% backset) as compared to a typical LOF mash pH of about 3 in the absence of backset.

### Example 6

Ethanol shake flask fermentations were completed using three levels of a GC 106 acid protease enzyme (available from Genencor) or elevated urea levels to confirm that assimilable nitrogen is a limiting factor in LOF based fermentation medium.

Two LOF mash batches, each having 23.1% w/w solids (DB) and 16.1 DE, were prepared in duplicate by grinding 912 grams of LOF (prepared as described in Example 1) (652 g starch) through a 0.75 mm screen and then combining it with 2588 mL DI water with agitation in a tared beaker. 451 μL Spezyme Fred L and 0.474 g of CaCl₂ were added. The temperature was raised to 90° C., held at that temperature for 25 minutes, and then cooled to 60° C. The pH was adjusted to 5 to 5.2 with sulfuric acid. 771 μL of glucoamylase (Distillase L-400) was then added. 3.2 g Wyeast nutrient was dissolved in 50 mL water and 42.8 mL was added to the LOF. 3.88 mL of a 0.2 g/mL urea solution was added (1.2 mg urea per gram of starch) and 1.37 mL of a 10 mg/mL V50 antibiotic solution were added to the mash. The two mash batches were combined and 600 g was dispensed into each of 9 sterilized 1-L shake flasks. A 0.25 g/mL yeast solution was prepared by adding 10 g yeast to 37 mL sterile buffer. A protease solution was prepared by a 1:100 dilution of GC106. The fermentations were prepared as indicated in Table 6A, below, where 2.32 ml of the yeast solution was added to each fermentation, and 0.8 ml of a 0.2 g/mL urea solution was added to fermentation 5 (about 2.4 mg total urea per gram starch). Fermentations 2, 3, 4 and 5 were run in duplicate. The fermentations were placed on a rotary shaker at 125 rpm and were maintained at 32° C. Each flask was sampled twice per day and analyzed for pH, ethanol (by HPLC), carbohydrate (by HPLC), yeast count and viability. Ethanol titer is reported in Table 6B, ethanol productivity is reported in table 6C, carbohydrate utilization is reported in Table 6D and fermentation pH is reported in Table 6E. Fermentation impurity content at 66 hours elapsed fermentation time (in g/L) is reported in Table 6F.

TABLE 6A
Fermentation Composition

| Composition | GC106 | Urea |
|---|---|---|
| LOF Control (Ferm. 1) | — | — |
| LOF + 0.128 SAPU GC106/g starch (Ferm. 2) | 1.46 mL | — |
| LOF + 0.064 SAPU GC106/g starch (Ferm. 3) | 0.73 mL | — |
| LOF + 0.032 SAPU GC106/g starch (Ferm. 4) | 0.36 mL | — |
| LOF + 2X urea (Ferm. 5) | — | 0.8 mL |

TABLE 6B
Ethanol Titer (g ethanol/L)

| Ferm | 18 Hours | 26 Hours | 42 Hours | 50 Hours | 66 Hours |
|---|---|---|---|---|---|
| 1 | 50 | 63 | 85 | 95 | 109 |
| 2 | 55 | 72 | 101 | 112 | 120 |
| 3 | 53 | 70 | 97 | 108 | 118 |
| 4 | 51 | 67 | 93 | 104 | 118 |
| 5 | 60 | 74 | 104 | 115 | 116 |

TABLE 6C
Ethanol Productivity (g/L/hr)

| Ferm | 18 Hours | 26 Hours | 42 Hours | 50 Hours | 66 Hours |
|---|---|---|---|---|---|
| 1 | 2.78 | 2.42 | 2.02 | 1.9 | 1.65 |
| 2 | 3.06 | 2.77 | 2.4 | 2.24 | 1.82 |
| 3 | 2.94 | 2.69 | 2.31 | 2.16 | 1.79 |
| 4 | 2.83 | 2.58 | 2.21 | 2.08 | 1.79 |
| 5 | 3.33 | 2.85 | 2.48 | 2.3 | 1.76 |

Ethanol yield (g ethanol/g starch) at 66 hours was 0.43, 0.47, 0.47, 0.47 and 0.47 for fermentations 1-5, respectively.

TABLE 6D
Carbohydrate Utilization^{a}

| Ferm | 18 Hours | 26 Hours | 42 Hours | 50 Hours | 66 Hours |
|---|---|---|---|---|---|
| 1 | 142 | 116 | 72 | 49 | 24 |
| 2 | 130 | 113 | 37 | 19 | 6 |
| 3 | 138 | 104 | 46 | 25 | 5 |
| 4 | 138 | 108 | 55 | 35 | 7 |
| 5 | 119 | 93 | 33 | 12 | 6 |

^{a}Carbohydrate utilization is reported as the carbohydrate concentration remaining in solution in g/L.

TABLE 6E
Fermentation pH

| Ferm | 18 Hours | 26 Hours | 42 Hours | 50 Hours | 66 Hours |
|---|---|---|---|---|---|
| 1 | 3.13 | 3.13 | 3.11 | 3.14 | 3.21 |
| 2 | 3.11 | 3.07 | 3.09 | 3.17 | 3.32 |
| 3 | 3.14 | 3.12 | 3.11 | 3.12 | 3.33 |
| 4 | 3.14 | 3.11 | 3.11 | 3.12 | 3.3 |
| 5 | 3.16 | 3.12 | 3.1 | 3.11 | 3.32 |

TABLE 6F
Fermenter Broth Analysis (g/L) at 66 Hours
Fermentation Time

| Analyte | Ferm. 1 | Ferm. 2 | Ferm. 3 | Ferm. 4 | Ferm. 5 |
|---|---|---|---|---|---|
| DP3+ | 3.1 | 2.35 | 2.15 | 2.7 | 1.95 |
| DP2 | 1.85 | 1.75 | 1.79 | 1.81 | 1.68 |
| Glucose | 18.9 | 1.04 | 1.09 | 1.61 | 1.23 |
| Total Carbs | 23.9 | 5.15 | 5.03 | 6.12 | 4.87 |
| Succinic A. | 2.2 | 1.9 | 2.0 | 2.1 | 1.9 |
| Lactic A. | 0.24 | 0.19 | 0.22 | 0.22 | 0.24 |
| Glycerol | 9.5 | 9.9 | 10.0 | 9.6 | 11.0 |
| Acetic A. | 0.34 | 0.27 | 0.4 | 0.4 | 0.16 |
| Propionic A. | 0.3 | 0.3 | 0.3 | 0.4 | 0.4 |
| Ethanol | 109.9 | 119.8 | 119.3 | 118.2 | 118.1 |

Example 6 results demonstrate that both acid protease and elevated urea were successful in allowing essentially complete utilization of fermentable carbohydrates in LOF. LOF supplementation with acid protease at levels of 0.032 SAPU, 0.064 SAPU and 0.128 SAPU per gram of starch all enabled fermentations to proceed to completion. Ethanol production rates were slightly faster with increasing enzyme dosage. Ethanol yields were consistent regardless of enzyme dosage. Fermentations containing 24 mg urea per gram of starch preformed similarly with fermentations performed with acid protease. Neither the acid protease or the supplemental urea significantly affected pH during fermentation. The highest dose of protease achieved a slightly lower pH than the other fermentations early in the fermentation.

### Example 7

A yellow number 2 corn mash composite was prepared and supplemented in separate flasks with 500 ppm, 1,000 ppm, 2,000 ppm and 4,000 ppm L-lysine in order to evaluate the effect of lysine concentration on yeast count and ethanol yield.

Two mash batches, each having 30.7% w/w solids (DS), were prepared in duplicate by combing 1,328 grams ground yellow number 2 corn (837 g starch) with 2522 mL tap water with agitation in a tared beaker. 1.67 mL Spezyme Fred L (0.002 mL per gram of starch) 0.25 g of CaCl₂ (20 ppm) were added. The temperature was raised to 90° C., held at that temperature for 25 minutes. The batches were combined and cooled to about 40° C. The pH was adjusted to 5 to 5.2 with sulfuric acid. 1.330 mL tap was added to dilute the mash composition to about 22.43% starch (DS). 2.01 mL of glucoamylase (Distillase L-400) was then added. A g/mL yeast suspension was prepared by adding 11.7 g yeast to 44 mL sterile buffer. The yeast suspension was put into a shaker at 31° C. for at least 15 minutes. 8.8 grams Wyeast nutrient was added to 137 water and heated to dissolve. The yeast nutrient was added to the mash. 12.3 mL of 0.2 g/mL urea solution was added to the mash (about 1.5 mg urea per gram of starch). 3.75 mL of 10 mg/mL V10 antibiotic was added to the mash. 30 mL of the yeast suspension was added to the mash. The mash was then divided into 10 flasks. Flasks 1 and 2 (fermentation 1) contained no added lysine; Flasks 3 and 4 (fermentation 2) contained 500 ppm added lysine; Flasks 5 and 6 (fermentation 3) contained 1,000 ppm added lysine; Flasks 7 and 8 (fermentation 4) contained 2,000 ppm added lysine; and Flasks 9 and 10 (fermentation 5) contained ppm added lysine. The flasks were incubated at 31° C. for 50 hours and sampled twice per day and analyzed for pH, ethanol (by HPLC), carbohydrate (by HPLC), yeast count and viability. Ethanol titer is reported in Table 7A, ethanol productivity is reported in table 7B, carbohydrate utilization is reported in Table 7C, fermentation impurity content at 64 hours elapsed fermentation time (in g/L) is reported in Table 7D, and ethanol yield on a starch basis is reported in Table 7E.

TABLE 7A
Ethanol Titer (g ethanol/L)

| Ferm | 17 Hours | 24 Hours | 40 Hours | 48 Hours | 64 Hours |
|---|---|---|---|---|---|
| 1 | 55.8 | 71.1 | 99.6 | 109.3 | 111.1 |
| 2 | 59.1 | 75.9 | 105.1 | 111.8 | 111.4 |
| 3 | 53.5 | 69.6 | 97.2 | 105 | 110.8 |
| 4 | 51.8 | 67.3 | 94.4 | 97.5 | 111.3 |
| 5 | 51.5 | 66.7 | 92.9 | 102.3 | 110.1 |

TABLE 7B
Ethanol Productivity (g/L/hr)

| Ferm | 17 Hours | 24 Hours | 40 Hours | 48 Hours | 64 Hours |
|---|---|---|---|---|---|
| 1 | 3.28 | 2.96 | 2.49 | 2.28 | 1.74 |
| 2 | 3.48 | 3.16 | 2.63 | 2.33 | 1.74 |
| 3 | 3.15 | 2.9 | 2.43 | 2.19 | 1.73 |
| 4 | 3.05 | 2.8 | 2.36 | 2.03 | 1.74 |
| 5 | 3.03 | 2.78 | 2.32 | 2.13 | 1.72 |

TABLE 7C
Carbohydrate Utilization^{a}

| Ferm | 17 Hours | 24 Hours | 40 Hours | 48 Hours | 64 Hours |
|---|---|---|---|---|---|
| 1 | 110.8 | 82.2 | 25.2 | 7 | 6.8 |
| 2 | 105.8 | 74.3 | 16.4 | 6.8 | 6.4 |
| 3 | 117.1 | 86.1 | 30.7 | 8.3 | 6.3 |
| 4 | 123.6 | 96.6 | 42.1 | 24.6 | 8 |
| 5 | 124.3 | 97.5 | 43.1 | 26.7 | 8.5 |

^{a}Carbohydrate utilization is reported as the carbohydrate concentration remaining in solution in g/L.

TABLE 7D
Fermenter Broth Analysis (g/L) at 66 Hours
Fermentation Time

| Analyte | Ferm. 1 | Ferm. 2 | Ferm. 3 | Ferm. 4 | Ferm. 5 |
|---|---|---|---|---|---|
| DP4+ | 2.23 | 2.31 | 3.47 | 3.11 | 3.77 |
| DP3+ | 0.97 | 0.92 | 0.99 | 1.09 | 1.08 |
| DP2 | 1.99 | 1.8 | 1.54 | 2.42 | 2.42 |
| Glucose | 0.35 | 0.31 | 0.33 | 0.82 | 0.63 |
| Fructose | 1.28 | 1.08 | 0.98 | 0.58 | 0.57 |
| Total Carbs | 6.81 | 6.4 | 6.32 | 8.01 | 8.45 |
| Lactic A. | 0 | 0 | 0 | 0 | 0 |
| Glycerol | 10.88 | 9.97 | 9.73 | 9.13 | 8.96 |
| Acetic A. | 0.35 | 0.34 | 0.32 | 0.7 | 0.85 |
| Ethanol | 111.1 | 111.41 | 110.8 | 111.4 | 110.1 |

TABLE 7E
Ethanol Yield on a Starch Basis (g ethanol per g starch).

| Time | Ferm. 1 | Ferm. 2 | Ferm. 3 | Ferm. 4 | Ferm. 5 |
|---|---|---|---|---|---|
| 48 hours | 0.446 | 0.456 | 0.428 | 0.409 | 0.417 |
| 64 hours | 0.453 | 0.455 | 0.452 | 0.454 | 0.449 |

Experimental results indicate that a mash having a L-lysine concentration of about 500 ppm accelerated ethanol productivity and provided higher yield over the control at fermentation times of less than 64 hours. Fermentation mashes with L-lysine concentrations of 1000 ppm or greater were rate impaired, but eventually reached completion by 64 hours where there was no significant difference in ethanol yield between the test and control fermentations. Yeast cell size was larger by a factor of about 2× in the fermentations containing 1000 ppm or more L-lysine. Yeast viability in all fermentations was greater than about 97% in all fermentations until 40 hours at which time the 2000 and 4000 ppm L-lysine mashes dropped to about 75% viability.

### Example 8

A LOF fermentation trial was done to simulate a 17-hour fermentation fill and to determine if a pH drop occurs that affects fermentation.

Two 14-liter New Brunswick fermenters were used. One was modified for liquifaction of the LOF mash using steam in a coil for temperature adjustment and the other was used as a fermenter.

Mash was made using 3800 g of LOF (87.5% DS), 3180 g water, and 3316 g thin stillage obtained from a commercial ethanol production facility. The LOF was tested and determined to contain 70.4% starch on an as is basis (80.5% on a dry (anhydrous) basis).

Liquifaction was done by adding 1.76 g Spezyme Xtra amylase (from Genencor) to the mash and heating at 90° C. for 50 minutes. The Spezyme Xtra dosage was 0.00066 ml/g starch. The pH was then lowered to less than 4.5 with HCL to inactivate the enzyme. After 10 minutes, the temperature was lowered to 45° C. and the pH was adjusted up to 5.0 with NaOH. A sample was then taken and tested for pH and quantitatively analyzed by HPLC.

A yeast propagation was made using 130 g of liquified mash, 65 g of water, 0.6 g of 20% urea, 0.18 g of Ethanol Red yeast, 0.04 g of G-zyme glucoamylase, and 2 ml of antibiotic. About 0.004 g of urea per gram of starch was added to provide FAN of about 0.002 g N/g starch. Propagation was carried out at 31° C. and an agitation rate of 200 rpm for 16 hours. A sample was taken at end of propagation for pH, HPLC and yeast count analysis. The 16 hour yeast count was 9.38×10⁸.

An initial fill of about 2000 ml of liquified mash was placed in the fermenter resulting in about 20% of the total fermentation volume. The mash volume was sufficient to reach the lowest agitator blade. The yeast from the propagation vessel and 41.5 ml of 20% urea was then added to the fermenter. The ratio of the yeast propagation volume to fermenter volume was about 0.019. About 1.5 ml of G-zyme glucoamylase was added to the fermenter with the yeast and about 1.5 ml was added when the fermenter was almost full. About 155 g of liquifaction mash was added every 15 minutes for a total of 11 hours using a peristaltic pump. The fermentation liquid was aerated as the fermenter filled. Samples were taken when the fermenter was ½ full, finished filling, and 24 hrs, 28 hours, 32 hours, 48 hours, and 52 hours after yeast addition. The samples were tested for pH and carbohydrate profile. Yeast count was completed on the 24-hour sample (13 hours after filling) and was found to be 8.95×10⁸. The results are presented in Table 8A where concentrations are in grams per liter.

TABLE 8A

| | End of | End of | Propagation | Fermentation |
|---|---|---|---|---|
| | liquifaction | liq. feed | flask | ½ full |
| pH | 5.0 | 4.8 | 4.3 | 4.2 |
| DP4+ | 194.4 | 186.9 | 22.3 | 66.7 |
| DP3 | 27.9 | 26.3 | 3.5 | 18.8 |
| DP2 | 24.2 | 21.6 | 3.2 | 52.2 |
| Dextrose | 29.0 | 24.7 | 1.1 | 124.1 |
| Fructose | 3.6 | 3.4 | 0.6 | 1.0 |
| Lactic | 1.2 | 1.0 | 1.8 | 1.4 |
| Glycerol | 5.3 | 4.6 | 5.6 | 5.7 |
| Acetic | 0.9 | 0.9 | 0.4 | 1.0 |
| Propionic | 0.2 | 0.2 | — | 0.2 |
| EtOH | 0.0 | 0.0 | 59.1 | 10.0 |

| | Full | 24 hr | 28 hr | 32 hr | 48 hr | 52 hr | 56 hr |
|---|---|---|---|---|---|---|---|
| pH | 4.3 | 4.1 | 4.1 | 4.1 | 4.2 | 4.1 | 4.2 |
| DP4+ | 91.2 | 59.2 | 47.3 | 41.5 | 8.2 | 7.3 | 6.8 |
| DP3 | 29.6 | 6.2 | 6.4 | 6.6 | 3.7 | 3.3 | 3.1 |
| DP2 | 39.5 | 35.2 | 17.3 | 11.9 | 6.7 | 7.1 | 7.2 |
| Dextrose | 69.2 | 4.6 | 14.8 | 3.9 | 0.0 | 0.0 | 0.0 |
| Fructose | 1.0 | 0.9 | 1.0 | 0.9 | 1.0 | 1.0 | 1.0 |
| Lactic | 1.4 | 1.6 | 1.6 | 1.6 | 1.7 | 1.7 | 1.6 |
| Glycerol | 6.3 | 11.2 | 11.8 | 12.4 | 15.2 | 15.2 | 15.3 |
| Acetic | 0.9 | 0.9 | 0.7 | 0.9 | 1.1 | 1.1 | 1.1 |
| Propionic | 0.2 | 0.3 | 0.3 | 0.3 | 0.4 | 0.4 | 0.4 |
| EtOH | 18.2 | 80.9 | 91.5 | 99.1 | 130.5 | 134.9 | 134.4 |

Overall, the fermentation the pH during fill and fermentation remained above 4.0 and was acceptable. The Ethanol reached 130 g/L at 48 hours after the yeast were added (37 hours after fill), and 134 g/L at 52 hours (time final) after yeast addition. The overall rate was 2.59 g/L/hr.

### Example 9

Comparative LOF and standard corn fermentation trials were done in a 950,000 gallon (3,591,000 L) fermenter to evaluate industrial scale LOF fermentation as compared to standard corn feed fermentation. A standard corn fermentation had been previously done in the fermentation equipment.

LOF was prepared by fractionating standard corn feed in a Buhler-L apparatus wherein the ratio of LOF to HOF was 71:29. The LOF had an average starch content of 78.8%, an oil content of 1.38% and a moisture content of 9.2%. The LOF was milled in a hammer mill. The screening results are reported in Table 9A.

TABLE 9A
Milled screening results with results reported as percent
retained.

| | #12 screen | #16 screen | #20 screen | Pan |
|---|---|---|---|---|
| Standard | 2.96 | 5.01 | 31.05 | 61.83 |
| Corn | | | | |
| LOF | 3.66 | 6.29 | 33.1 | 57.73 |

The fermentation tankage was emptied as much as practical prior to introducing LOF into the fermentation process in order to clear standard corn from the process. A mash content of about 90-95% LOF and 5-10% standard corn resulted with the standard corn being introduced primarily from a combination of tank heels and standard corn backset.

LOF and standard corn mashes were separately prepared. To provide a common basis for fermentation evaluation, LOF and standard corn mashes having equivalent starch content were prepared. The standard corn was determined to have a starch content of 73% at 33.5% DS. The DS for the LOF was lowered to about 31% in order to achieve an equivalent starch loading. The composition of the LOF and standard corn mashes is reported in Table 9B.

TABLE 9B

| | LOF | Standard Corn |
|---|---|---|
| LOF | 31.8% | — |
| Standard Corn | — | 33.5% |
| Backset | 34% | 34% |
| Alpha-amylase g/kg | 0.13 | 0.12 |
| starch | | |

Liquifaction was done by first passing the mashes through a jet to a temperature of 108° C. and a residence time of about 7 to 10 minutes. The pH of both mashes pH was adjusted and controlled to 5.7 going into liquefaction. For standard corn, pH was controlled by adding ammonium hydroxide at an unknown rate corresponding to a 28% open control valve. For LOF, pH was also controlled by adding ammonium hydroxide, but the control valve was 14% open. LOF therefore resulted in reduced ammonium hydroxide usage, estimated to be 42% less than the usage required to maintain the standard corn mash at pH 5.7.

The mashes were cooled to about 90° C. Additional Spezyme Xtra enzyme was added at a dosage of 0.161 ml/g starch to the standard corn mash and 0.179 ml/g starch to the LOF mash. Residence time was about 1.5 hours.

For LOF as compared to standard corn, alpha-amylase enzyme loading was increased 12% by volume in the mash and 17% by volume during liquifaction (after the jet), for an average increase in alpha-amylase enzyme of 15%. This represents about a 13% increase in enzyme on a starch basis, or slightly less if the decrease in ammonia usage is accounted for.

The LOF liquifaction performance was satisfactory, with a slurry DE of 6.6 (versus about 6.0 for standard corn), and a liquifaction DE of 14.6 (versus about 12.0 for standard corn). This indicates that liquifaction enzyme usage could be cut back to usage levels similar to the standard corn process.

During LOF fermentation GA enzyme levels were increased by 2.3%, urea levels increased by 42%, and virginiamycin levels were held constant as indicated in Table 9C.

TABLE 9C

| | LOF | Standard Corn |
|---|---|---|
| Glucoamylase (g/kg | 0.81 | 0.79 |
| starch) | | |
| Added FAN (g N/kg | 2.12 | 1.49 |
| starch (as urea) | | |
| Virginiamycin | 2 ppm | 2 ppm |
| Backset | 34% | 34% |

The results for the LOF and standard corn are reported in Tables 9D and 9E, respectively, in w/w %.

TABLE 9D
LOF Fermentation results

| Variable | 6 hr | 14 hr | 21 hr | 28 hr | 35 hr |
|---|---|---|---|---|---|
| pH | 5.14 | 4.22 | 4.22 | 4.4 | 4.43 |
| DP4 | 12.27 | 9 | 7.05 | 5.08 | 2.41 |
| DP3 | 1.67 | 0.64 | 0.25 | 0.27 | 0.21 |
| Maltose | 5.36 | 6 | 3.64 | 1.25 | 0.44 |
| Glucose | 8.07 | 3.99 | 1.62 | 1.97 | 1.7 |
| Lactic Acid | 0.087 | 0.13 | 0.14 | 0.15 | 0.16 |
| Glycerol | 0.65 | 0.98 | 1.16 | 1.24 | 1.3 |
| Acetic Acid | 0.046 | 0.021 | 0.026 | 0.04 | 0.053 |
| Ethanol | 1.43 | 5.08 | 8.43 | 10.34 | 12 |
| Incremental | — | — | 0.48 | 0.27 | 0.24 |
| Ethanol Rate | | | | | |
| Starch Conversion | 0.09 | 0.34 | 0.57 | 0.71 | 0.83 |

| Variable | 41 hr | 46.5 hr | 51 hr | 59.5 hr |
|---|---|---|---|---|
| pH | 4.5 | 4.5 | 4.8 | 4.82 |
| DP4 | 1.48 | 0.96 | 0.83 | 0.78 |
| DP3 | 0.14 | 0.089 | 0.08 | 0.079 |
| Maltose | 0.42 | 0.43 | 0.44 | 0.44 |
| Glucose | 1.24 | 0.72 | 0.54 | 0.52 |
| Lactic Acid | 0.15 | 0.15 | 0.15 | 0.15 |
| Glycerol | 1.35 | 1.38 | 1.39 | 1.39 |
| Acetic Acid | 0.05 | 0.051 | 0.053 | 0.055 |
| Ethanol | 13 | 13.5 | 13.8 | 13.7 |
| Ethanol Rate | 0.17 | 0.1 | 0.052 | — |
| Starch Conversion | 0.89 | 0.93 | 0.94 | 0.94 |

TABLE 9E
Standard Corn Fermentation results

| Variable | 10 hr | 18 hr | 25 hr | 32 hr |
|---|---|---|---|---|
| pH | 4.5 | 4.35 | 4.31 | 4.36 |
| DP4 | 10.49 | 8.25 | 5.72 | 3.24 |
| DP3 | 0.39 | 0.2 | 0.14 | 0.16 |
| Maltose | 4.35 | 3.15 | 1.17 | 0.6 |
| Glucose | 8.42 | 6.44 | 4.95 | 3.89 |
| Lactic Acid | 0.11 | 0.11 | 0.12 | 0.14 |
| Glycerol | 0.81 | 1 | 1.2 | 1.24 |
| Acetic Acid | 0.044 | 0.025 | 0.031 | 0.047 |
| Ethanol | 2.79 | 5.4 | 8.22 | 10.1 |
| Ethanol Rate | — | — | 0.403 | 0.272 |
| Starch Conversion | — | — | 0.58 | 0.72 |

| Variable | 39 hr | 45 hr | 55 hr |
|---|---|---|---|
| pH | 4.33 | 4.41 | 4.5 |
| DP4 | 1.74 | 1.25 | 1.07 |
| DP3 | 0.094 | 0.076 | 0.065 |
| Maltose | 0.52 | 0.54 | 0.5 |
| Glucose | 2.44 | 1.13 | 0.35 |
| Lactic Acid | 0.13 | 0.13 | 0.13 |
| Glycerol | 1.31 | 1.37 | 1.39 |
| Acetic Acid | 0.054 | 0.054 | 0.055 |
| Ethanol | 11.7 | 12.7 | 13.3 |
| Ethanol Rate | 0.22 | 0.165 | 0.06 |
| Starch Conversion | 0.83 | 0.90 | — |

The LOF fermentation proceeded at a maximum measured rate of 0.478 wt % Ethanol/hr, as compared to a rate wt %/hr for the standard corn. The result was a 19% increase in peak fermentation rate. Average fermentation rates also increased from 0.212 to 0.260 wt %/hr, for a 23% increase in average rate. The fermentation endpoint concentration was increased while fermentation endpoint time was decreased. The observed ethanol titer increase was 4.5% and ethanol titer increase based on mash DS basis was 10%.

Based on experimental evidence to date, ethanol yield on a starch basis was observed to be greater for LOF than for standard corn. The rise in yield was calculated by two measures. The yield increase (i.e. gal ethanol/bushel corn) was calculated indirectly in two ways. First, by the amount of ethanol produced per DS compared to control. Second, by % fermentables observed to be consumed, as measured by HPLC, for LOF versus the control. Both calculations indicated a 1.8-2.7% increase in ethanol yield, averaging out at 2.2%. The increase might be rationalized in terms of a separation of fermentable starch towards the LOF fraction, and more of the less fermentable and/or protein-bound starch being removed with the HOF fraction.

The ethanol produced from LOF was recovered by distillation. Whole corn stillage was not effectively purged from the beer well resulting in an additional 200,000 gallons of whole corn stillage being combined with 500,000 gallons of LOF based stillage. The result was a mixed stillage containing about 68% LOF and 32% whole corn was sent to the still.

No marked changes in distillation performance were noticed in terms of temperatures or flow rates. However, it was noted that the amount of fusel oil in the side draw was low and the draw was exceptionally clear. The ethanol was analyzed for fusel oil content. The method involved collecting an ethanol distillate sample from the side draw and pouring 100 mL of the sample into a 250 mL graduated cylinder. An equal volume of saturated salt (NaCl) solution was added to the cylinder and mixed. After 10 minutes the volume of light oils at the top of the cylinder was measured and divided by 100 to provide an approximate fusel oil concentration. The ethanol prepared had no measurable fusel oil as compared to prior art whole corn processes that typically contain about 5-10% measured fusel oil. Low fusel oil concentration can lead to the ability to cut down on the fusel draw from the distillation column, thereby decreasing the moisture of the 95% ethanol sent to the molecular sieves.

Centrifugation of the LOF reboiler bottoms resulted in a torque decreased by about 10% as compared to standard corn. As compared to DDG generated from standard corn, the DDG generated from LOF provided about an 8% decrease in steam usage required to achieve a baseline moisture content of 64%. It is believed that reduced centrifuge torque and dryer steam usage is an indication of the reduced LOF solids loading.

### Example 10

Two ethanol fermentations were done with LOF at a high weight solids loading of 33.8 weight percent in order to evaluate final ethanol titer.

For each fermentation, in a tared 1 L flask an LOF mash weighing 250 grams and containing 33.8% w/w solids was prepared by combining LOF (prepared as described in Example 1) with a mixture of 70% DI water and 30% backset obtained from a dry corn milled ethanol plant. The LOF contained 84.6 wt % starch on an anhydrous basis. 0.047 mL of Spezyme Xtra was added and the mash was held at 90-95° C. for 1 hour with frequent shaking to mix the ingredients. The pH was then adjusted to 4.6 with sulfuric acid and the mash was cooled to less than about 33° C. To the mash was added 1.79 mL of urea to provide 5 mg of urea per gram of starch, 0.1 mL of V50 antibiotics (available from North American Bioproducts Corp. and containing a blend of virginiamycin, penicillin, and streptomycin) and 0.1074 mL G-zyme 480. The flask contents were then dried and the dried contents were weighed. The contents were then redissolved in DI water added to reach the initial total weight.

A yeast propagation mash was prepared by combining 24 g LOF, 24 g backset as described above, 57 g DI water and 0.014 mL Spezyme in a 250 mL shake flask. The pH was checked and adjusted to 5.6 with NaOH if necessary. The mash was held at 90-95° C. for 1 hour with frequent shaking to mix the ingredients. The pH was then adjusted to 4.6 with sulfuric acid and the mash was cooled to less than about 33° C. To the mash was added 0.215 mL of 20% urea providing 2.5 mg urea/g starch), 0.027 g G-zyme 480, and 0.04 mL V50 antibiotic and 0.105 g active dry yeast (Fermentis Ethanol Red). The yeast propagation mixture was incubated in a shaker at 31° C. overnight. The total cell count after incubation was 7.4×10⁸ cell/mL for fermentation 1 and 7.7×10⁸ cell/mL for fermentation 2.

To the 250 g of LOF mash was added 10 mL of the propagated yeast. The fermentations were run in a shaker flask at 31° C. in a water bath. Samples were collected at 16, 24, 40 and 48 hours and analyzed by HPLC with the results reported below in grams per liter in Table 10a for fermentation 1 and Table 10b for fermentation 2 where dextrose prod is the total dextrose possible from breakdown of carbohydrates plus that calculated from the ethanol produced at that point.

TABLE 10a

| | 16 hrs. | 24 hrs. | 40 hrs. | 48 hrs. |
|---|---|---|---|---|
| DP3 | 4.25 | 3.76 | 1.96 | 1.66 |
| DP2 | 7.35 | 4.39 | 5.85 | 6.13 |
| Dextrose | 84.6 | 56.3 | 7.52 | 4.45 |
| Fructose | 3.09 | 3.08 | 1.85 | 1.26 |
| Succinic Acid | 0.83 | 0.91 | 1.31 | 1.0 |
| Glycerol | 12.3 | 14.2 | 15.5 | 14.8 |
| Acetic Acid | 0.06 | 0.34 | 0.71 | 0.74 |
| Propionic Acid | 0.21 | 0.30 | 0.51 | 0.55 |
| Ethanol | 69.5 | 96.5 | 134.2 | 128.5 |
| Carbohydrates | 99.3 | 67.5 | 17.2 | 13.5 |
| Carb. as dextrose | 100.6 | 68.4 | 18.1 | 14.4 |
| Dextrose prod. | 236.5 | 257.2 | 280.6 | 265.8 |
| Potential Ethanol | 111.7 | 123.6 | 137.7 | 130.3 |
| Ethanol rate | 4.34 | 4.02 | 3.35 | 2.68 |
| (g/L/hr) | | | | |
| pH | 4.01 | 4.10 | 4.62 | — |

TABLE 10b

| | 16 hrs. | 24 hrs. | 40 hrs. | 48 hrs. |
|---|---|---|---|---|
| DP3 | 0 | 2 | 1.13 | 1.1 |
| DP2 | 6.08 | 3.57 | 5.13 | 6.06 |
| Dextrose | 71.5 | 57.4 | 5.9 | 4.65 |
| Fructose | 2.83 | 2.76 | 1.90 | 1.46 |
| Succinic Acid | 0.8 | 0.99 | 1.08 | 1.15 |
| Glycerol | 11.8 | 15.2 | 16.1 | 16.8 |
| Acetic Acid | 0.08 | 0.46 | 0.85 | 0.95 |
| Propionic Acid | 0.19 | 0.29 | 0.6 | 0.69 |
| Ethanol | 67.7 | 105.3 | 143.7 | 150.8 |
| Carbohydrates | 82.6 | 84.4 | 18.8 | 17.6 |
| Carb. as dextrose | 81.1 | 66.2 | 14.7 | 14 |
| Dextrose prod. | 213.5 | 272.2 | 295.8 | 309 |
| Potential Ethanol | 100.8 | 131.4 | 145.6 | 152.4 |
| Ethanol rate | 4.23 | 4.39 | 3.59 | 3.14 |
| (g/L/hr) | | | | |
| pH | 4.17 | 4.24 | 4.84 | — |

Fermentations 1 and 2 were essentially complete at 40 hours where total ethanol titers of 134.2 g/L (fermentation 1) and 143.7 g/L (fermentation 2) resulted.

When introducing elements of the present invention or the preferred embodiments(s) thereof, the articles “a”, “an”, “the” and “said” are intended to mean that there are one or more of the elements. The terms “comprising”, “including” and “having” are intended to be inclusive and mean that there may be additional elements other than the listed elements.

In view of the above, it will be seen that the several objects of the invention are achieved and other advantageous results attained.

As various changes could be made in the above compositions and processes without departing from the scope of the invention, it is intended that all matter contained in the above description and shown in the accompanying drawings shall be interpreted as illustrative and not in a limiting sense.

What is claimed is:

1. A fermentation process for producing ethanol from whole corn, the process comprising:
fractionating the whole corn;
separating the fractionated corn into a low oil fraction and a high oil fraction, the low oil fraction comprising starch;
forming a slurry comprising water and the low oil fraction;
liquifying the low oil fraction to form a mash;
forming a fermentation medium comprising the mash, an added source of free amino nitrogen, yeast and backset, the fermentation medium containing at least about 1.2 milligrams of added free amino nitrogen per gram of starch and the backset constituting at least about 25 percent by volume of the fermentation medium;
saccharifying and fermenting the fermentation medium to produce a crude fermentation composition comprising ethanol and carbohydrates; and
recovering the ethanol from the crude fermentation composition.

2. The process of claim 1 wherein the low oil fraction comprises, on an anhydrous basis, less than about 3 percent by weight total oil, at least about 72 percent by weight starch, from about 5 percent by weight to about 11 percent by weight crude protein, and less than about 20 percent by weight non-fermentables.

3. The process of claim 1 wherein the fermentation medium contains from about 1.2 to about 6 mg of added free amino nitrogen per gram of starch.

4. The process of claim 1 wherein the pH of the fermentation medium is not adjusted.

5. The process of claim 1 wherein the ethanol concentration in the crude fermentation composition is from about 120 to about 150 grams of ethanol per liter.

6. The process of claim 5 wherein the crude fermentation composition ethanol concentration is reached in from about 40 to about 55 hours of elapsed fermentation time.

7. The process of claim 1 wherein the crude fermentation composition further comprises distillers dried grain with solubles, wherein the distillers dried grain with solubles are recovered from the crude fermentation composition and dried in a drier and wherein the distillers dried grain with solubles yield, calculated as the ratio of the weight of distillers dried grain with solubles produced to weight of low oil fraction is from about 0.15 to about 0.25.

8. The process of claim 1 wherein the whole corn is yellow number 2 corn, highly fermentable corn, high oil corn, high lysine corn or mixtures thereof.

9. The process of claim 1 wherein the source of free amino nitrogen is urea.

10. The process of any claim 1 wherein the fermentation medium further comprises a solvent extracted high oil fraction and the weight ratio of the solvent extracted high oil fraction to the low oil fraction is from about 5:95 to about 40:60.

11. The process of claim 1 wherein the net energy input required to produce a liter of ethanol is reduced from 1% to 10% as compared to a reference fermentation process, the reference fermentation process being devoid of the low oil fraction and instead comprising whole corn, but otherwise identical to the low oil fraction fermentation process.

12. The process claim 1 wherein at least 91% by weight of the starch is converted to ethanol.

13. The process claim 1 wherein the crude fermentation composition further comprises fusel oil in a concentration of less than about 0.5 grams/L.

14. The process of claim 1 wherein the crude fermentation composition further comprises fusel oil and the weight ratio of ethanol to fusel oil in the crude fermentation composition is at least about 100:1.

15. A fermentation process for producing ethanol from whole corn, the process comprising:
fractionating the whole corn;
separating the fractionated whole corn into a low oil fraction an a high oil fraction, the low oil fraction comprising, on an anhydrous basis, less than about 3 percent by weight total oil, at least about 72 percent by weight starch, from about 5 percent by weight to about 11 percent by weight crude protein, and less than about 20 percent by weight non-fermentables;
forming a slurry comprising water and the low oil fraction;
liquifying the low oil fraction to form a mash;
forming a fermentation medium comprising the mash, an added source of free amino nitrogen and yeast, wherein the added free amino nitrogen content is at least about 1.2 milligrams of free amino nitrogen per gram of starch in the mash;
saccharifying and fermenting the fermentation medium to produce a crude fermentation composition comprising ethanol and carbohydrates; and
recovering the ethanol from the crude fermentation composition.

16. The process claim 15 wherein the fermentation medium contains from about 1.2 to about 6 mg of added free amino nitrogen per gram of starch.

17. The process claim 15 wherein the pH of the fermentation medium is not adjusted.

18. The process of claim 15 wherein the ethanol concentration in the crude fermentation composition is from about 120 to about 150 grams of ethanol per liter.

19. The process of claim 15 wherein the crude fermentation composition ethanol concentration is reached in from about 40 hours to about 55 hours of elapsed fermentation time.

20. The process of claim 15 wherein the crude fermentation composition further comprises distillers dried grain with solubles, wherein the distillers dried grain with solubles are recovered from the crude fermentation composition and dried in a drier and wherein the distillers dried grain with solubles yield, calculated as the ratio of the weight of distillers dried grain produced to weight of low oil fraction is from about 0.15 to about 0.25.

21. The process of claim 15 wherein the whole corn is yellow number 2 corn, highly fermentable corn, high oil corn, high lysine corn or mixtures thereof.

22. The process claim 15 wherein the source of free amino nitrogen is urea.

23. The process of claim 15 wherein the fermentation medium further comprises backset and wherein the backset comprises at least about 25 percent by volume of the fermentation medium.

24. The process of claim 15 wherein the fermentation medium further comprises a solvent extracted high oil fraction and the weight ratio of the solvent extracted high oil fraction to the low oil fraction is between about 5:95 and about 40:60.

25. The process of claim 15 wherein the net energy input required to produce a liter of ethanol is reduced from 1% to 10% as compared to a reference fermentation process, the reference fermentation process being devoid of the low oil fraction and instead comprising whole corn, but otherwise identical to the low oil fraction fermentation process.

26. The process of claim 15 wherein at least about 91% of the starch is converted to ethanol.

27. The process of claim 15 wherein the crude fermentation composition further comprises fusel oil in a concentration of less than about 0.5 grams/L.

28. The process of claim 15 wherein the crude fermentation composition further comprises fusel oil and the weight ratio of ethanol to fusel oil in the crude fermentation composition is at least about 100:1.

29. A distillers dried grain with solubles prepared from a fermentation process, the process comprising:
fractionating whole corn;
separating the fractionated corn into a low oil fraction and a high oil fraction, the low oil fraction comprising starch;
forming a slurry comprising water and the low oil fraction;
liquifying the low oil fraction to form a mash;
forming a fermentation medium comprising the mash and yeast;
saccharifying and fermenting the fermentation medium to produce a crude fermentation composition comprising crude dried distillers grain with solubles;
recovering the crude dried distillers grain with solubles from the crude fermentation composition; and
recovering distillers dried grain with solubles from the crude dried distillers grain with solubles and drying the distillers dried grain with solubles in a drier, wherein the distillers dried grain with solubles comprises greater than 35 weight percent total protein and wherein the dried distillers grain with solubles yield, calculated as the ratio of the weight of dried distillers grain with solubles produced, on an anhydrous basis, to weight of starch in the mash is less than 0.25.

30. The distillers dried grain with solubles of claim 29 wherein the low oil fraction further comprises acid detergent fiber, wherein the acid detergent fiber concentration, on an anhydrous basis, is less than about 7 wt %.

31. The distillers dried grain with solubles of claim 29 wherein the low oil fraction further comprises neutral detergent fiber, wherein the neutral detergent fiber concentration, on an anhydrous basis, is less than about 12 wt %.

32. The distillers dried grain with solubles of claim 29 wherein the low oil fraction further comprises ash, wherein the ash concentration, on an anhydrous basis, is less than 1.2 wt %.

33. The distillers dried grain with solubles of claim 29 wherein the distillers dried grain with solubles yield, calculated as the ratio of the weight of distillers dried grain produced to weight of low oil fraction is from about 0.15 to about 0.25.

34. The distillers dried grain with solubles of claim 29 wherein the whole corn is yellow number 2 corn, highly fermentable corn, high oil corn, high lysine corn or mixtures thereof.

35. The distillers dried grain with solubles of claim 29 wherein the low oil fraction comprises, on an anhydrous basis, less than about 3 percent by weight total oil, at least about 72 percent by weight starch, from about 5 percent by weight to about 11 percent by weight crude protein, and less than about 20 percent by weight non-fermentables.
